(19) United States
(12) Patent Application Publication
(10) Pub. No.: US 2010/0113494 A1
(43) Pub. Date: May 6, 2010

(54) 13,13a-Dihydroberberine Derivatives For Use In Pharmaceutical Compositions

(75) Inventors: Lihong Hu, Shanghai (CN); Jia Li, New District Shanghai (CN); Jingya Li, New District Shanghai (CN); Hankun Zhang, New District Shangai (CN); Zhe Cheng, New District Shanghai (CN); Jiming Ye, New South Wales (AU); David E. James, New South Wales (AU); Edward W. Kraegen, New South Wales (AU)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: SHANGHAI INSTITUTE OF MATERIA MEDICA, CHINESE ACADEMY OF SCIENCES, Pudong New District Shanghai (CN)

(21) Appl. No.: 12/443,401

(22) PCT Filed: Sep. 30, 2007
(86) PCT No.: PCT/CN2007/002882

§ 371 Date: Jan. 4, 2010

(30) Foreign Application Priority Data
Sep. 30, 2006 (CN) 200610140429.6

(51) Int. Cl. A61K 31/4375

(52) U.S. Cl. 514/280; 514/279; 514/287

(57) ABSTRACT

The present invention provides 13,13a-dihydroberberine derivatives or their physiologically acceptable salts represented by the following formula, pharmaceutical compositions comprising the same, and uses thereof. The 13,13a-dihydroberberine derivatives have an activity of promoting glucose absorption in muscle cells, and the whole animal tests show that the present compounds have effects on improving glucose-tolerance and insulin-resistance, facilitating weight loss, relieving fatty liver and the like. Thus, the present compounds can be used in treating diabetes mellitus, adiposity, fatty liver and complications thereof induced by insulin resistance.

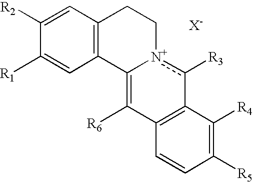

### FIELD OF THE INVENTION

The present invention generally relates to the fields of pharmaceutical chemistry and therapeutic treatment of diseases. More particularly, the present invention relates to pharmaceutical compositions of 13,13a-dihydroberberine derivatives and the use thereof as an insulin sensitizer.

### BACKGROUND OF THE INVENTION

Diabetes mellitus is a group of clinical metabolic diseases induced by the genetic and environmental interactions, and involves a series of disorders of carbohydrate, protein, fat, water and electrolyte metabolisms due to absolute or relative deficiencies in insulin secretion or reduced sensitivity of the target tissues to insulin. Clinically, diabetes mellitus is mainly characterized by hyperglycemia and may lead to damages to multiple organic systems as time goes on, and acute metabolic disorder such as ketoacidosis etc. may occur in serious conditions and stress. The incidence of severe complications such as cardiovascular diseases, kidney damage, diabetic angiopathy and peripheral vascular disease leading to blindness and gangrene etc. in patients suffering from diabetes mellitus is significantly higher than that in persons free of diabetes mellitus. Therefore, diabetes mellitus and complications thereof are the cause of serious public health problems that threaten human health throughout the world.

Diabetes mellitus is mainly classified into two types, namely type 1 diabetes mellitus (insulin-dependent diabetes mellitus, IDDM) and type 2 diabetes mellitus (non-insulin-dependent diabetes mellitus, NIDDM). Presently, over 95% of diabetic patients suffer from type 2 diabetes mellitus. With the improvement of living conditions, the incidence of diabetes mellitus is increasing due to aging, obesity, and participating in unhealthy life style choices such as eating an unhealthy diet and lacking exercise. According to the statistics, the worldwide number of the diabetic patients has exceeded 190 million, and it is estimated that this figure will rise to 330 million in 2025.

Type 1 diabetic patients have abnormal response to stimuli of environment factors, in particular such as viral infection or chemical toxic substance, due to genetic susceptibility determined by HLA-D gene in the short arm of chromosome 6, which destroys the pancreatic beta-cells through direct or indirect autoimmune response and hence results in insulin deficiency. These patients have the clinical features of quick onset, obvious symptoms such as polyphagia, polyuria, polydipsia and weigh loss etc., and the tendency of having ketoacidosis, and have to rely on insulin therapy to maintain their lives.

Type 2 diabetes mellitus is also strongly affected by genetic and environmental factors, and shows significant heterogeneity, that is, its pathogenesis is varied and complicated, and there are great differences among type 2 diabetic patients. Generally, the reasons that cause type 2 diabetes mellitus can be attributed to the relative deficiencies in insulin secretion and/or insulin resistance. A series of studies on type 2 diabetic patients, in particular obese diabetic patients, have identified that the insulin resistance is a key factor during the occurrence and development of type 2 diabetes mellitus. Therefore, according to the results obtained from the studies on the insulin signaling pathway in adipose and muscle cells, it is currently a focus and a major tendency of the development of new drugs for type 2 diabetes mellitus to design and develop insulin sensitizer so as to improve the condition of insulin resistance.

At present, the oral drugs for diabetes treatment in clinic can be classified into three main types including insulin secretagogues, insulin sensitizers and carbohydrate modulator.

Sulfonylureas insulin secretagogues: Sulfonylureas lower blood glucose levels in human body by stimulating the release of insulin from the pancreatic beta-cells. Such type of drugs includes Glibenclamide, Glipizide, Gliquidone, Gliclazide, Glimepiride etc. Sulfonylureas are the first-line medications for non-obese type 2 diabetic patients. However, all of sulfonylureas run the risk of causing hypoglycemia, i.e. abnormally low and dangerous levels of blood glucose.

Non-sulfonylureas insulin secretagogues: Non-sulfonylureas that are highly related in terms of mechanism of action are the rapid-acting prandial insulin releasers. More particularly, they stimulate insulin secretion from the pancreatic beta-cells by increasing the concentration of Ca²⁺ in the cells through Ca²⁺ inflowing caused by closing the ATP depended K⁺ channels on the membranes of the pancreatic beta-cells. Such drugs including Repaglinide, Nateglinide etc. are mainly used as a diet regulator to adjust high blood glucose levels after meals. However, they can not improve insulin resistance, and may cause the patients to have temporal anaphylactic response.

Thiazolidinediones insulin sensitizers: Thiazolidinediones (TZDs) are a class of high selective peroxisome proliferator activated receptor γ (PPARγ) agonists, which may improve insulin resistance and correct abnormal glucose and lipid metabolism. The mechanism of action of TZDs is to control blood glucose levels of human body effectively by increasing insulin sensitivity in its targeted tissues. Such drugs include Troglitazone hydrochloride (it has been withdrawn from market because of safety problems in term of hepatotoxicity, and the reason for causing such a side effect is still not found out), Rosiglitazone, Pioglitazone. The main side effects of the drugs are weight gain, edema and so on.

Biguanides insulin sensitizers: Biguanides cannot promote insulin secretion. Instead, their mechanism of action is to lower blood glucose levels by increasing glucose uptake of the peripheral tissues such as muscle, promoting the anaerobic glycolysis of tissues, improving the glucose absorption of tissues such as muscle, suppressing hepatic gluconeogenesis and decreasing the incidence of hyperglycemia in the case of diabetes mellitus. Biguanides can improve glycometabolism and cause weight loss without affecting blood serum insulin levels.

Therefore, there is no risk of lowering blood glucose levels after administration of Biguanides for the patients with normal blood glucose levels. Thus, Biguanides are the first-line drugs for obese diabetic patients. Such drugs include Phenethylbiguanide (which has been withdrawn from market due to safety problems), Metformin hydrochloride, Metformin hydrochloride sustained-release tablets etc. However, a large dose of Biguanides is required to be administrated to improve the insulin sensitivity. For example, the oral dose of Metformin hydrochloride is 1500 mg per day (mg/d), which is the upper limit of dose in China. The accompanying side effects are mainly gastrointestinal symptoms such as nausea, diarrhea and cramps etc., and the old with impaired heart or rental function are at a risk of lactic acidosis when taking such drugs.

Drugs for reducing the absorption of carbohydrates are mainly α-glucosidase inhibitors and aldose reductase inhibitors. α-Glucosidase inhibitors may prevent the increase of blood glucose levels after meals by suppressing the decomposition and digestion of carbohydrates and disaccharides, and delaying and reducing the absorption of glucose in the upper segment of small intestine through competitively suppressing the activity of α-glucosidase which take part in the degradation of carbohydrates in the villi of small intestines. They may be used as the first-line drugs with an appropriate diet and exercise, or they may be used in combination with sulfonylureas, biguanides and/or insulin. This type of drugs includes Acarbose, Voglibose and Miglitol etc. Their main adverse effects are gastrointestinal upsets such as abdominal discomfort and flatulence etc. Aldose Reductase (AR) is a key rate-limiting enzyme in the polyol pathway. AR inhibitors have been a new focus in the research fields of diabetic therapy since 1970s.

A large number of animal experiments and clinical researches indicate that AR inhibitors can normalize effectively the impaired ployol pathway in organisms so as to prevent and/or delay diabetic complications. Tolrestat is a drug for diabetic therapy which was developed by Wyeth Pharmaceuticals and marketed in Ireland in 1989. However, it was not approved by the Food and Drug Administration (FDA) because of causing visual impairment and renal failure, and was withdrawn from market in 1996. Epalrestat is another drug for diabetic therapy which is marketed in Japan recently. However, it also has some similar adverse effects.

Therefore, the current drugs for the treatment of type 2 diabetes mellitus cannot sufficiently control blood glucose levels and are not effective for all diabetic patients. Moreover, each type of drugs has more or less adverse effects. Thus, it is still a research highlight currently to develop new drugs which have new mechanism and are safe and effective for the treatment of type 2 diabetes mellitus.

Berberine, also called umbellatine, is an isoquinoline alkaloid represented by the following formula, which is a major active component in such plants as Coptidis chinensis Franch., Berberis amurensis Rupr., Berberis sargentiana Schneid and Nandina domestica etc. Clinically, berberine is mainly used to treat diarrhea caused by bacteria with the most important advantage of having little side effects. Berberine has been used as a broad spectrum antibiotic for many years, and is effective on inhibition and elimination of various Gram-positive and Gram-negative bacteria, fungi, moulds, viruses, protozoa and nematode.

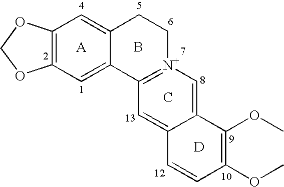

Structure of Berberine

Since 1950s, a large number of animal experiments and clinical trials have been carried out by some specialists, researchers and clinicians with berberine hydrochloride which is widely used in clinic, and the results showed that berberine are effective on the therapy of diabetes mellitus and their complications besides being antibiotic. Some research literatures are summarized in the following Table I:

TABLE I
Summary of animal experiments and clinical trials on treating diabetes mellitus with berberine

| Authors | Results of animal studies and clinical trials | Literature |
|---|---|---|
| Qiming CHEN, | Berberine possesses advantages of both sulfonylureas and | Acta |
| Mingzhi XIE | biguanides. It can lower blood glucose levels of both normal mice | Pharmaceutica |
| (Institute of | and alloxan-induced diabetic mice, and antagonize the increase of | Sinica, 1986, |
| Materia Medica, | blood glucose levels induced by exogenous glucose or | 21(6): 401-406. |
| Chinese Academy | epinephrine. It has powerful effect on lowering blood glucose | |
| of Medical | levels and long action time lasting up to 5-6 hours. Berberine can | |
| Sciences) | also improve abnormal blood coagulation and prevent | |
| | atherosclerosis. It had a significant effect on lowering blood | |
| | glucose at a dose of 40 mg/kg. | |
| Yanxie NI, et. al. | 60 cases of type 2 diabetic patients were treated with berberine | Journal of |
| (The 208th hospital | and the treatment was lasted for 1-3 months. The results showed | Integrated |
| of PLA) | that the fasting blood glucose decreased averagely by 5.1 mmol/L | Traditional and |
| | and the efficiency was 90%. It was found by pathologic | Western |
| | examinations that berberine could promote the regeneration and | Medicine, 1988, |
| | functional recovery of pancreatic beta-cells. No abnormality was | 8(12): 711-713. |
| | observed for all patients in the hepatic and renal functions and | |
| | hemogram during the treatment and no obvious adverse effect | |
| | was found. | |
| Guoying LIU | 30 cases of type 2 diabetic patients were treated with berberine | New use of old |
| (Pengze traditional | and the efficiency was 90%. Berberine was significantly effective | drug, 1992, 1: 3-4. |
| Chinese medical | for the patients with light or moderate diabetes mellitus. In | |
| hospital, Jiujiang, | addition, it also showed effects on lowering blood pressure and | |
| Jiangxi) | blood fat, and preventing infection and the occurrence of | |
| | complication etc. | |
| Mianrong YU, et. | 75 cases of type 2 diabetic patients were treated with berberine | Beijing Medical |
| al. (Builder | and the efficiency was 69.33%. Three diabetic symptoms, i.e. | Journal, 1994, |
| hospital of Beijing) | polyuria, polydipsia and polyphagia, disappeared for all patients | 16(2): 117. |
| | within two weeks after administration of berberine accompanying | |
| | the decrease of blood glucose. The diabetic patients have been | |
| | treated with berberine and observed for 4 years, and none of them | |
| | was observed with adverse effects or hypoglycemia regardless of | |
| | dose. In addition, 11 patients had normal blood glucose with | |
| | reduced dose more than 3 years. | |
| Faguang HU, et. al. | 60 cases of type 2 diabetic patients were treated with berberine | The Practical |
| (The Shanxuan | and the efficiency was 90%. Berberine had a mild and lasting | Journal of |
| people's hospital, | effect on lowering blood glucose, making the blood glucose start | Integrating of |
| Henan) | to lower after about 2 weeks, and achieving its maximum curative | Chinese with |
| | effect within 8 weeks. For all patients, berberine made the | Modern Medicine, |
| | diabetic symptoms disappearing or being relieved and showed | 1995, 8(6): |
| | good effects on common diabetic complications. In addition, 4 | 358-359. |
| | patients with overweight recovered their normal weights. No | |
| | toxic and/or adverse effects were observed. | |
| Jiaqing ZHANG | Berberine has a certain effect on increasing insulin sensitivity and | Liaoning Journal |
| (Changhai hospital, | can improve insulin resistance with an insulin action index (IAI) | of Practical |
| the 2nd military | similar to metformin. | Diabetology, |
| surgeon college) | | 1999, 1(4): 56-57. |
| Changshan LIU, et | Renal failure often occurs to most of diabetic patients with the | Chinese Journal of |
| al. (Weifang | deterioration of conditions. Berberine, a traditional Chinese drug, | Diabetes, 1996, |
| medical college, | has a significant effect on reversing diabetic renal failure and | 4(3): 163-166. |
| Shandong) | inhibiting the activities of the polyol and kidney aldose reductases | |
| | of diabetic mice to lower urine protein. | |
| Jinhua TONG | 49 cases of type 2 diabetic patients were treated with berberine | New Journal of |
| (Sanitation School | and the efficiency was 63.3%. Berberine can suppress | Traditional |
| of Fengtai, Anhui) | glyconeogenesis and glycogenolysis, slow down insulin | Chinese Medicine, |
| | metabolism and promote the sensitivity of peripheral tissue cells | 1997, 29(3): |
| | to insulin by lowering the activity of the sympathetic nerve and | 33-34. |
| | inhibiting the adrenal cortical function. It can also promote the | |
| | regeneration and functional recovery of pancreatic cells. | |
| Huading YU, | The effect of berberine on treating non-insulin dependent diabetic | Medical Journal of |
| Laiwen YAN | patients was evaluated. The patients were divided randomly into 2 | Communications, |
| (Shanghai seaman | groups, and each group had 30 persons. One group took berberine | 1995, 9(4): 45. |
| hospital) | and another took Diamicron. After 4 weeks treatment, the | |
| | variations of the concentrations of blood glucose and blood serum | |
| | insulin were compared between the two groups, and the results | |
| | showed that the two groups had similar curative effects and no | |
| | severe adverse effects were observed. | |
| Yunfei ZHANG | For 20 cases of type 2 diabetic patients, their conditions were not | Chinese Journal of |
| (Hangzhou | improved significantly after they took oral anti-diabetic drugs for | Integrated |
| sanatorium of Air | lowering blood glucose such as D860 and glibenclamide etc. | Traditional and |
| Force) | Then, they were treated with berberine. The fasting blood glucose | Western |
| | levels of all patients were lowered to normal levels 3 months | Medicine, 1999, |
| | later. The effect of berberine on lowering blood glucose was | 19(9): 567. |
| | dependent on dose, and no side effects such as hypoglycemia | |
| | were observed. | |
| Hong ZHU, Wei | 112 cases of type 2 diabetic patients were treated with berberine | Journal of Jining |
| DAI (Affiliated | and the efficiency was 90%, which showed that berberine had a | Medical College, |
| hospital of Jining | good effect on lowering blood glucose. Specifically, berberine | 1999, 22(3): 67. |
| medical college) | had mild and lasting effect of lowering blood glucose without | |
| | severe toxicity, side effects and damages to liver and renal | |
| | function, and had good curative effect on common complications | |
| | of diabetes mellitus. | |
| Litian SHI, et al. | 68 cases of type 2 diabetic patients were treated with berberine. | Shanxi Clinical |
| (Taiyuan | The blood glucose levels of the patients were lowered | Medicine, 2000, |
| commercial staff | significantly, and the total efficiency was 86.8%. The action | 9(3): 181-182. |
| hospital) | mechanism of berberine on lowering blood sugar was multiple. | |
| | The blood serum insulin levels of the patients increased | |
| | significantly after the treatment. | |
| Dongkou WANG | 57 cases of type 2 diabetic patients were treated with a second | Zhejiang Journal |
| (Taizhou Health | generation sulfonylureas drug alone or in combination with | of Integrated |
| center, Jiangsu) | metformin. However, secondary failure occurred 3 years later. | Traditional |
| | Thus, berberine was taken additionally. The blood glucose levels | Chinese and |
| | of the patients were lowered more or less 1 month later, and total | Western |
| | efficiency was 81%. | Medicine, 2002, |
| | | 12(1): 50. |
| Jiandong JIANG et | Berberine can interact with insulin receptors and increase | CN01121906.8 |
| al. | significantly the expressions of insulin receptor genes and | |
| | peroxisome proliferator activated receptor genes. Therefore, | |
| | berberine can be used as an insulin sensitizer to treat type 2 | |
| | diabetes mellitus alone or in combination with insulin or other | |
| | type 2 diabetic drugs. | |
| Kaimin WU | Highly soluble berberines such as berberine sulfate and berberine | WO03090749A1, |
| | phosphate etc. are easily dissolved in water, which may improve | CN1771944A |
| | the absorption of the drugs in organism and thus enhance | |
| | significantly their curative effect. Thus, they can be used to treat | |
| | and prevent diabetes mellitus or its complications. | |

Recently, berberine has been widely used to treat type 2 diabetes mellitus in clinic. It was found in an initial research that the antihyperglycemic activity of berberine was in association with its activities of resisting hyperglycemic hormones and promoting the regeneration and functional recovery of pancreatic beta-cells (Hongyan ZHENG, Weiren XU, Chinese herbal medicine. 2004, 35: 708-711). Such research showed that 5-100 μmol of berberine could increase the glucose consumption in HepG2 cells by 32-60%, but it did not stimulate βTC3 cells to secrete insulin. So it was concluded that the antihyperglycemic activity of berberine acted by increasing the glucose consumption of liver cells instead of stimulating to secrete insulin, i.e., the antihyperglycemic activity of berberine acted through liver cells without depending on insulin.

Juan WANG (Juan WANG, China and foreign medical Journal. 2004, 2(12): 65-66) reported that it was observed clinically that the blood glucose levels decreased and the blood serum insulin levels increased when berberine was used to treat diabetes mellitus, which indicated that berberine could promote the regeneration and functional recovery of pancreatic beta-cells besides its resistance to the hyperglycemic hormones. Berberine can also inhibit gluconeogenesis and improve glycolysis, thereby lowering the blood glucose. Thus, the antihyperglycemic activity of berberine belongs to the field of insulin sensitizer. Berberine also has positive effects on preventing diabetes mellitus complications because of its activities of anti-hypertension, anti-hyperlipidemia and anti-infection.

Up to now, the antidiabetic mechanism of berberine has not been definitely expounded, although it has a significant effect on treating diabetes mellitus. Recently, Guangde YANG et al. from Xi'an Jiaotong University reported that palmatine hydrochloride, an analogue of berberine, could be used to treat type 2 diabetes mellitus (CN1582930A). They also synthesized various analogues of berberine and tetrahydro-berberine, and evaluated their antihyperglycemic activity using cell-membrane chromatography and alloxan-induced diabetic mice (Bioorganic & Medicinal Chemistry Letters, 2006, 16: 1380-1383).

It was found in our research that berberine had a similar effect to clinical diabetic drugs of Biguanides, and could improve the uptake and absorption of glucose in peripheral tissues of the human body (e.g. muscles) so as to lower blood glucose levels of diabetic patients and achieve the treatment purpose. So, berberine belongs to non-insulin dependent glucose absorption enhancer in the peripheral tissues of the human body. It has been reported in the literatures (Klip, A., et al., Endocrinology 1992, 130, 2535-2544; Yamamoto, N. et al, Anal. Bio. 2006, 351, 139-145) that metformin hydrochloride could significantly increase the glucose uptake in human body muscle tissues in a model test to evaluate glucose uptake activity in L6 muscle cells. In a model test on the glucose transport in L6 muscle cells, the transport rate of 3-methyl glucose in L6 muscle cells increased by about 0.5 times with 400 μM metformin hydrochloride. Berberine hydrochloride could achieve the same effect with less dosage than metformin in a model test on the glucose transport in L6 muscle cells in vitro.

In our study, the glucose transport rate in L6 muscle cells increased by about 3 times with 10 μM berberine hydrochloride. In a further glucose absorption model test in L6 muscle cells, the glucose absorption in L6 muscle cells increased by more than 2 times with 5 berberine hydrochloride in 20 mM glucose medium. It was found that berberine had little effect on insulin signaling pathway, but significantly reinforced the activities of AMPK and p38 MAPK kinase with an immunoblotting assay of L6 muscle cells. The improvement in glucose absorption induced by insulin was inhibited obviously, while that induced by berberine was not affected after the treatment with wortmannin, a specific inhibitor of insulin signaling pathway. The glucose absorption induced by berberine was obviously lowered after the treatment with AMPK and p38 MAPK specific inhibitors (Compound C and SB202190, respectively).

The results of a further immunoblotting test showed that AMPK was located in the upstream of p38 MAPK in the cell signaling pathway affected by berberine. The results of the above tests indicated that AMPK was a key target protein by which berberine improved the glucose absorption. Through high performance liquid chromatography, it was found that the AMP:ATP ratio in cells increased significantly, indicating a variation of the energy metabolism levels in cells, which is the key factor of AMPK activation. To sum up, our research showed that berberine improved the glucose absorption by changing the energy metabolism level in cells and thus activating AMPK protein instead of affecting insulin signaling pathway.

In general, berberine has a significant activity of treating diabetes mellitus. However, it has some disadvantageous features such as poor solubility and low oral bioavailability, etc., which restrict the further use of berberine on treating diabetes mellitus. Thus, it is necessary to extensively investigate the relationship between the structure and anti-diabetic activity of berberine, and find out berberine derivatives having better activity and absorption in vivo.

### SUMMARY OF THE INVENTION

The present invention is provided to solve the above problems.

An object of the present invention is to provide 13,13a-dihydroberberine derivatives or their physiologically acceptable salts represented by following Formula 1:

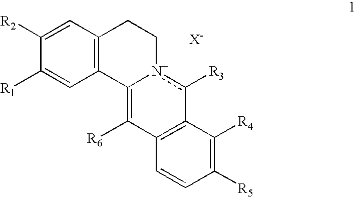

Wherein, “ [symbol] ” is a double bond or single bond;

R₁ and R₂ are each independently H, OH, C₁-C₄ alkoxyl or C₁-C₄ acyloxy, or R₁ and R₂ are combined into —O—CH₂—O—;

R₃ is H, OH, C₁-C₄ alkoxyl, C₁-C₄ acyloxy, C₁-C₄ alkyl, C₁-C₄ haloalkyl or an aryl;

R₄ and R₅ are each independently H, OH, C₁-C₄ alkoxyl or C₁-C₄ acyloxy, or R₄ and R₅ are combined into —O—CH₂—O—;

R₆ is H, OH, C₁-C₄ alkyl, C₁-C₄ haloalkyl, an aryl, C₁-C₄ alkoxyl or C₁-C₄ acyloxy.

Preferably, the 13,13a-dihydroberberine derivatives of the present invention are compounds 2-18 shown in the following Table II:

TABLE II

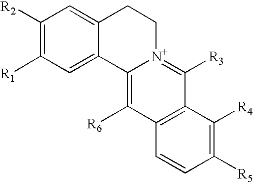

| compound | R₁ | R₂ | R₃ | R₄ | R₅ | R₆ | X |
|---|---|---|---|---|---|---|---|
| 2 | —O—CH₂—O— | | H | OCH₃ | OCH₃ | CH₃ | I |
| 3 | —O—CH₂—O— | | H | OCH₃ | OCH₃ | CH₃CH₂ | Br |
| 4 | —O—CH₂—O— | | H | OCH₃ | OCH₃ | CH₃CH₂CH₂ | I |
| 5 | —O—CH₂—O— | | H | OCH₃ | OCH₃ | CH₃CH₂CH₂CH₂ | I |
| 6 | —O—CH₂—O— | | H | OCH₃ | OCH₃ | ICH₂CH₂CH₂ | I |
| 7 | —O—CH₂—O— | | H | OCH₃ | OCH₃ | C₆H₅CH₂ | Cl |
| 8 | —O—CH₂—O— | | H | OCH₃ | OCH₃ | 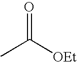 | Cl |
| 9 | —O—CH₂—O— | | H | OCH₃ | OCH₃ | 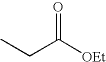 | Br |
| 10 | —O—CH₂—O— | | H | OCH₃ | OCH₃ | OH | Cl |
| 11 | —O—CH₂—O— | | H | OCH₃ | OCH₃ | OCH₃ | I |
| 12 | —O—CH₂—O— | | H | OCH₃ | OCH₃ | OCH₂CH₃ | Br |
| 13 | —O—CH₂—O— | | H | OCH₃ | OCH₃ | OCOCH₃ | Cl |
| 14 | —O—CH₂—O— | | H | OH | OCH₃ | H | Cl |
| 15 | —O—CH₂—O— | | H | OCOCH₃ | OCH₃ | H | Cl |
| 16 | —O—CH₂—O— | | H | OCOOCH₂CH₃ | OCH₃ | H | Cl |
| 17 | OH | OH | H | OCH₃ | OCH₃ | H | Cl |
| 18 | OH | OH | H | OCH₃ | OCH₃ | CH₃CH₂ | Cl |

Preferably, the 13,13a-dihydroberberine derivatives of the present invention are compounds 19-41 shown in the following Table III:

TABLE III

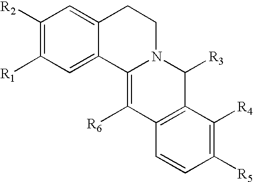

| com- | | | | | | |
|---|---|---|---|---|---|---|
| pound | R₁ | R₂ | R₃ | R₄ | R₅ | R₆ |
| 19 | —O—CH₂—O— | | H | OCH₃ | OCH₃ | H |
| 20 | —O—CH₂—O— | | CH₃CH₂ | OCH₃ | OCH₃ | H |
| 21 | —O—CH₂—O— | | PhCH₂ | OCH₃ | OCH₃ | H |
| 22 | —O—CH₂—O— | | HO | OCH₃ | OCH₃ | H |
| 23 | —O—CH₂—O— | | CH₃O | OCH₃ | OCH₃ | H |
| 24 | —O—CH₂—O— | | CH₃CH₂O | OCH₃ | OCH₃ | H |
| 25 | —O—CH₂—O— | | CCl₃ | OCH₃ | OCH₃ | H |
| 26 | —O—CH₂—O— | | CH₃COCH₂ | OCH₃ | OCH₃ | H |
| 27 | —O—CH₂—O— | | H | OCH₃ | OCH₃ | CH₃ |
| 28 | —O—CH₂—O— | | H | OCH₃ | OCH₃ | CH₃CH₂ |
| 29 | —O—CH₂—O— | | CH₃ | OCH₃ | OCH₃ | H |
| 30 | —O—CH₂—O— | | CH₃ | OCH₃ | OCH₃ | CH₃ |
| 31 | —O—CH₂—O— | | CH₃CH₂ | OCH₃ | OCH₃ | CH₃CH₂ |
| 32 | OH | OH | H | OCH₃ | OCH₃ | H |
| 33 | OH | OH | H | OH | OCH₃ | H |
| 34 | OH | OH | CH₃ | OCH₃ | OCH₃ | H |
| 35 | OH | OH | H | OCH₃ | OCH₃ | CH₃ |
| 36 | OH | OH | CH₃ | OCH₃ | OCH₃ | CH₃ |
| 37 | OAc | OAc | H | OCH₃ | OCH₃ | H |
| 38 | OAc | OAc | H | OAc | OAc | H |
| 39 | OCH₃ | OCH₃ | H | OCH₃ | OCH₃ | H |
| 40 | OCH₃ | OCH₃ | H | OCH₃ | OCH₃ | CH₂CH₃ |

| | | | | |
|---|---|---|---|---|
| 41 | —O—CH₂—O— | H | —O—CH₂—O— | H |

The 13,13a-dihydroberberine derivatives or their physiologically acceptable salts of the present invention can be synthesized by the following process:

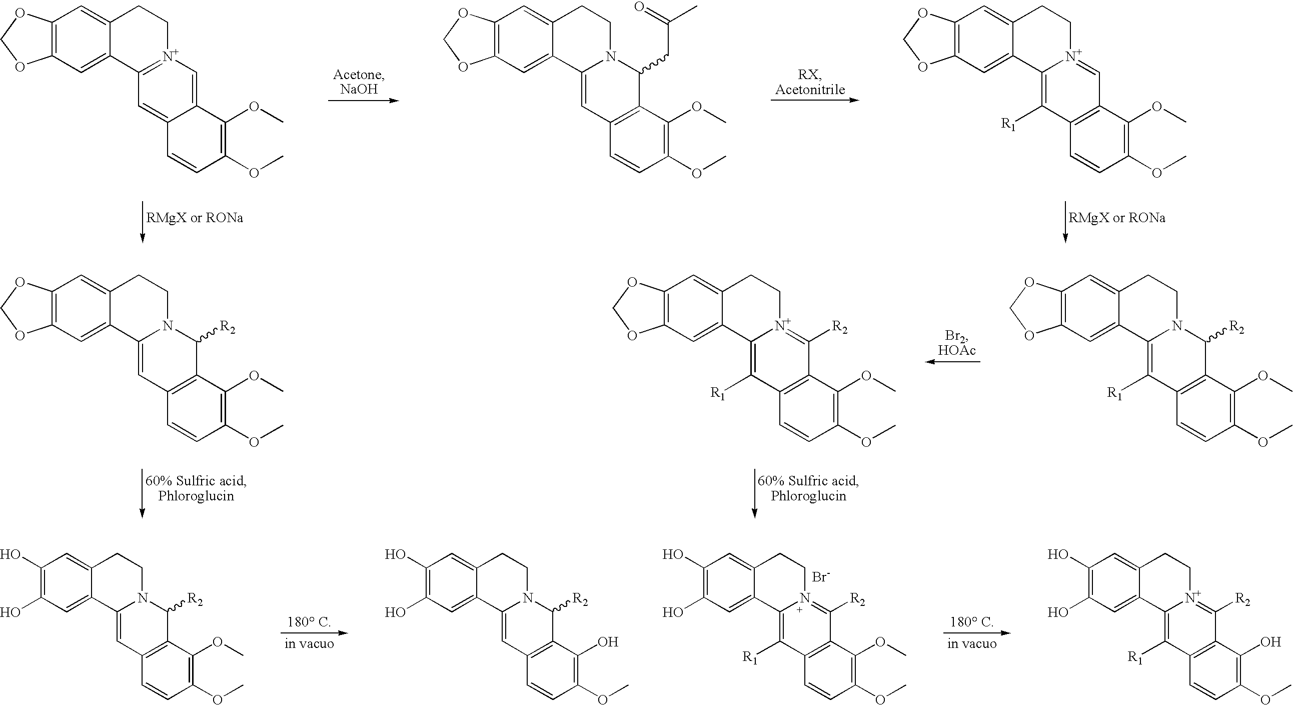

Berberine hydrochloride is dissolved in acetone and treated with a base such as NaOH to obtain acetonylberberine, wherein the 8-site of berberine is attacked by the α-site of acetone. Then, acetonylberberine reacts with a halohydrocarbon under heating to obtain 13-alkylberberine. 13-alkylberberine then reacts with a Grignard reagent to produce a 8,13-dialkyldihydroberberine derivatives. 13-alkylberberine can also react with sodium alcoholate anion under a strong base condition to form 8-alkoxyl-13-alkyldihydroberberine derivatives.

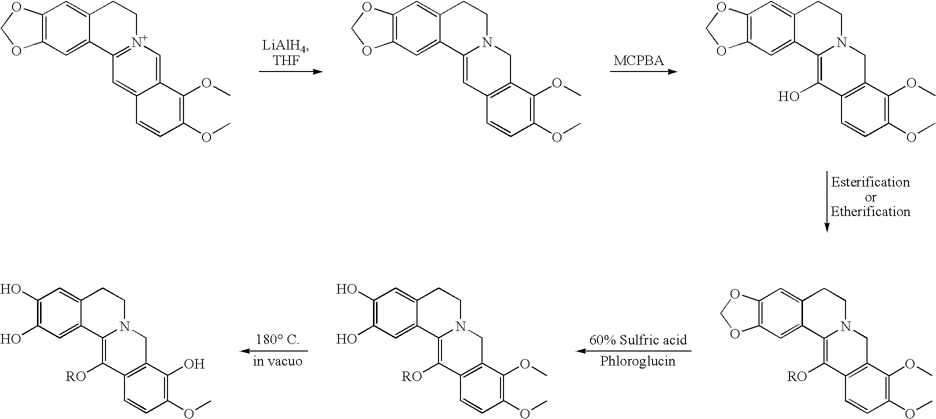

Berberine is reduced into a dihydroberberine with lithium-aluminum hydride in anhydrous tetrahydrofuran. The dihydroberberine is then oxidized with m-chloroperoxybenzoic acid (MCPBA) to obtain a 13-hydroxydihydroberberine. After oxidation, the 13-hydroxydihydroberberine is etherified or esterified to produce a series of 13-alkoxyl or 13-alkanoyloxy dihydroberberines.

Another object of the present invention is to provide a pharmaceutical composition containing a therapeutically effective amount of 13,13a-dihydroberberine derivatives or their physiologically acceptable salts.

Still another object of the present invention is to provide uses of the above 13,13a-dihydroberberine derivatives or their physiologically acceptable salts in preparing medicaments for treating diabetes mellitus, adiposity, fatty liver and their complications caused by insulin resistance.

Still another objection of the present invention is to provide uses of the pharmaceutical composition containing a therapeutically effective amount of 13,13a-dihydroberberine derivatives or their physiologically acceptable salts in preparing medicaments for treating diabetes mellitus, adiposity, fatty liver and their complications caused by insulin resistance.

### ADVANTAGEOUS EFFECTS

The present invention designs and synthesizes a series of 13,13a-dihydroberberine derivatives, which may promote glucose absorption in muscle cells. The animal tests showed that this series of compounds had effects on improving glucose tolerance and insulin resistance, facilitating weight loss and relieving fatty liver etc. Thus, these compounds can be used to treat diabetes mellitus, adiposity, fatty liver and their complications caused by insulin resistance. The compounds of present invention are easy to prepare and synthesize, and the materials thereof are abundant.

### BRIEF DESCRIPTION OF THE DRAWINGS

FIG. 1 is a graph illustrating the promotion effect of the compound according to the present invention at 5 M on glucose transport in L6 muscle cells.

FIG. 2A is a graph showing the intraperitoneal glucose tolerance test (ipGTT) after treating the obese mice with dihydroberberine derivative 19 and its sulfate salt for 2 weeks.

FIG. 2B is a graph showing the incremental areas under the intraperitoneal glucose tolerance test (ipGTT) curves after treating the obese mice with dihydroberberine derivative 19 and its sulfate salt for 2 weeks.

FIG. 3A is a graph showing the changes in the body weight of mice after the treatment with dihydroberberine derivative 19 and its sulfate salt for 2 weeks.

FIG. 3B is a graph showing the changes in visceral fat/body weight ratio of mice after the treatment with dihydroberberine derivative 19 and its sulfate salt for 2 weeks.

FIG. 4A is a graph showing the changes in the content of free fatty acid in blood plasma of mice after the chronic treatment with dihydroberberine derivative 19 and its sulfate salt for 2 weeks.

FIG. 4B is a graph showing the changes in the content of triglyceride in blood plasma of mice after the chronic treatment with dihydroberberine derivative 19 and its sulfate salt for 2 weeks.

### DETAILED DESCRIPTION

### Best Mode for Carrying Out the Invention

Hereinafter, the present invention will be described in more detail through the following examples. However, the present invention is not limited to or by the examples.

In the following preparation examples, ¹H-NMR measurement was carried out on a Varian Mercury AMX300 instrument, and MS determination was performed using a VG ZAB-HS or VG-7070 tape as well as Esquire 3000 plus-01005 instruments. All solvents were redistilled before use, and the used anhydrous solvents were subjected to being dried using standard methods. Unless otherwise specified, all reactions were carried out under argon atmosphere and tracked by TLC, and each product was washed with saturated sodium chloride solution and dried over anhydrous magnesium sulfate at post-processing. Unless otherwise stated, all products were purified by column chromatography using silica gels, and the used silica gel is GF₂₅₄ with a particle size of 200-300 mesh, which is commercially available form Qingdao Haiyang Chemical Co. Ltd or Yantai Yuanbo Silica Gel Co.

### PREPARATION EXAMPLES

### Preparation of Compound 2

8-acetonyl dihydroberberine (3 g) and methyl iodide were dissolved in 100 mL dichloromethane. The obtained reaction solution was heated to 100° C. under pressure and allowed to react for 3 hours. After the reaction finished, the solid by-product was filtered off, and the filtrate was evaporated to dryness under reduced pressure. The residue was recrystallized in methanol to obtain compound 2 (1.53 g, 48%).

Compound 2, C₂₁H₂₀INO₄, MW: 477, a yellow crystal, easily dissolved in a mixed solvent of chloroform and methanol.

¹H NMR (300 MHz, DMSO-d₆): δ 9.89 (1H, s, H-8), 8.20 (1H, d, J=9.0 Hz, H-12), 8.19 (1H, d, J=9.0 Hz, H-11), 7.48 (1H, s, H-1), 7.15 (1H, s, H-4), 6.18 (2H, s, —OCH₂O—), 4.80 (2H, m, H-6), 4.10 (3H, s, —OCH₃), 4.09 (3H, s, —OCH₃), 3.15 (2H, m, H-5), 2.92 (3H, s, —CH₃).

Preparation of Compound 3

8-acetonyl dihydroberberine (1.5 g) and ethyl bromide were dissolved in 100 mL dichloromethane. The obtained reaction solution was heated to 100° C. under pressure and allowed to react for 5 hours. After the reaction finished, the solid by-product was filtered off, and the filtrate was evaporated to dryness under reduced pressure. The residue was recrystallized in methanol to obtain compound 3 (0.83 g, 53%).

Compound 3, C₂₂H₂₂INO₄, MW: 491, a white crystal, easily dissolved in a mixed solvent of chloroform and methanol.

¹H NMR (300 MHz, DMSO-d₆): δ 9.90 (1H, s, H-8), 8.21 (2H, ABq, J=9.0 Hz, H-11 and 12), 7.30 (1H, s, H-1), 7.17 (1H, s, H-4), 6.19 (2H, s, —OCH₂O—), 4.80 (2H, m, H-6), 4.10 (3H, s, —OCH₃), 4.09 (3H, s, —OCH₃), 3.36 (2H, q, J=7.5 Hz, H-1′), 3.09 (2H, m, H-5), 1.47 (3H, t, J=7.5 Hz, H-2′).

Preparation of Compound 4

8-acetonyl dihydroberberine (0.5 g) and 1-propyl iodide (0.43 g) were dissolved in 50 mL dioxane. The obtained reaction solution was refluxed and allowed to react for 5 hours. After the reaction finished, the solid by-product was filtered off, and the filtrate was evaporated to dryness under reduced pressure. The residue was purified through a silica gel column (methanol:dichloromethane=1:10) to obtain compound 4 (0.25 g, 52%).

Compound 4, C₂₃H₂₄INO₄, MW: 505, a yellow crystal, easily dissolved in a mixed solvent of chloroform and methanol.

¹H NMR (300 MHz, DMSO-d₆): δ 9.96 (1H, s, H-8), 8.20 (2H, ABq, J=9.0 Hz, H-11 and 12), 7.23 (1H, s, H-1), 7.19 (1H, s, H-4), 6.21 (2H, s, —OCH₂O—), 4.80 (2H, m, H-6), 4.10 (3H, s, —OCH₃), 4.09 (3H, s, —OCH₃), 3.08 (2H, m, H-5), 1.83 (2H, m, H-1′), 1.10 (2H, m, H-2′), 1.05 (3H, t, J=7.0 Hz, H-3′).

Preparation of Compound 5

8-acetonyl dihydroberberine (1.0 g) and 1-butyl iodide (9.2 g) were dissolved in 50 mL acetonitrile. The obtained reaction solution was refluxed and allowed to react for 5 hours. After the reaction finished, the solid by-product was filtered off, and the filtrate was evaporated to dryness under reduced pressure. The residue was purified through silica gel column (methanol:chloroform=1:9) to obtain compound 5 (0.42 g, 43%).

Compound 5, C₂₄H₂₆INO₄, MW: 519, a yellow powder, easily dissolved in a mixed solvent of chloroform and methanol.

¹H NMR (300 MHz, DMSO-d₆): δ 9.89 (1H, s, H-8), 8.23 (2H, ABq, J=9.0 Hz, H-11 and 12), 7.31 (1H, s, H-1), 7.20 (1H, s, H-4), 6.11 (2H, s, —OCH₂O—), 4.80 (2H, m, H-6), 4.01 (6H, s, —OCH₃), 3.31 (2H, m, H-1′), 3.14 (2H, m, H-5), 1.82 (2H, m, H-2′), 1.47 (2H, m, H-3′), 0.95 (3H, t, J=7.5 Hz, H-4′).

Preparation of Compound 6

8-acetonyl dihydroberberine (2.65 g) and 1,3-diiodopropane (7.4 mL) were dissolved in 75 mL acetonitrile. The obtained reaction solution was refluxed and allowed to react for 6 hours. After reaction finished, the solid by-product was filtered off, and the filtrate was evaporated to dryness under reduced pressure. The residue was purified through silica gel column (methanol:chloroform=1:9) to obtain compound 6 (0.37 g, 8.7%).

Compound 6, C₂₃H₂₃I₂NO₄, MW: 631, a yellow powder, easily dissolved in a mixed solvent of chloroform and methanol.

¹H NMR (300 MHz, CDCl₃): δ 10.26 (1H, s, H-8), 8.00 (1H, d, J=9.0 Hz, H-11), 7.89 (1H, d, J=9.0 Hz, H-12), 7.09 (1H, s, H-1), 6.89 (1H, s, H-4), 6.10 (2H, s, —OCH₂O—), 5.08 (2H, m, H-6), 4.37 (3H, s, —OCH₃), 4.07 (3H, s, —OCH₃), 3.51 (2H, t, J=8 Hz, CH₂—Ar), 3.32 (2H, t, J=6 Hz, H-5), 3.23 (2H, t, J=5 Hz, CH₂—I), 2.30 (2H, m, CH_{2(propyl)}).

Preparation of Compound 7

8-acetonyl dihydroberberine (4 g) and sodium iodide (1.87 g) were dissolved in 50 mL of acetonitrile followed by adding 2 mL of benzyl chloride. The obtained reaction solution was heated to 80° C. and refluxed for 6 hours. Then, the reaction mixture was filtered and the filter cake was washed with acetonitrile. The combined filtrate was evaporated to remove acetonitrile under reduced pressure. The residual was purified through silica gel column (CHCl₃/CH₃OH=20:1) to obtain compound 7 (1.83 g, 45%).

Compound 7, C₂₇H₂₄ClNO₄, MW: 461, a brownish red amorphous powder, easily dissolved in a mixed solvent of chloroform and methanol.

¹H NMR (300 MHz, CDCl₃): δ 10.10 (1H, s, H-8), 7.63 (2H, dd, J=15.0 and 9.0 Hz, H-3′ and 5′), 7.32-7.23 (3H, m, H-1′, 4′ and 5′), 7.05 (2H, d, J=7.0 Hz, H-11 and 12), 6.90 (1H, s, H-1), 6.84 (1H, s, H-4), 5.93 (2H, s, —OCH₂O—), 4.98 (2H, m, H-6), 4.27 (3H, s, —OCH₃), 3.96 (3H, s, —OCH₃), 3.18 (2H, m, H-5).

Preparation of Compound 8

8-acetonyl dihydroberberine (4 g) and sodium iodide (1.87 g) were dissolved in 50 mL acetonitrile followed by adding 2 mL of ethyl chlorformate. The obtained reaction solution was heated to 80° C. and refluxed for 6 hours. Then, the reaction mixture was filtered and the filter cake was washed with acetonitrile. The combined filtrate was evaporated to remove acetonitrile under reduced pressure. The residual was purified through silica gel column (CHCl₃/CH₃OH=20:1) to obtain compound 8 (1.61 g, 40%).

Compound 8, C₂₃H₂₂ClNO₆, MW: 443, a yellow amorphous powder, easily dissolved in a mixed solvent of chloroform and methanol.

¹H NMR (300 MHz, CDCl₃): δ 10.82 (1H, s, H-8), 7.88 (1H, d, J=9.0 Hz, H-12), 7.73 (1H, d, J=9.0 Hz, H-11), 7.21 (1H, s, H-1), 6.89 (1H, s, H-4), 6.13 (2H, s, —OCH₂O—), 5.38 (2H, m, H-6), 4.40 (2H, q, J=7.5 Hz, H-2′), 4.08 (3H, s, —OCH₃), 4.02 (3H, s, —OCH₃), 3.38 (2H, m, H-5), 1.19 (3H, t, J=7.5 Hz, H-3′).

Preparation of Compound 9

8-acetonyl dihydroberberine (4 g) was dissolved in 30 mL acetonitrile followed by adding 2 mL of ethyl chloroacetate. The obtained reaction solution was heated to 80° C. and refluxed for 6 hours. Then, the reaction mixture was filtered and the filter cake was washed with acetonitrile. The combined filtrate was evaporated to remove acetonitrile under reduced pressure. The residual was purified through silica gel column (CHCl₃/CH₃OH=20:1) to obtain compound 9 (1.34 g, 35%).

Compound 9, C₂₄H₂₄ClNO₆, MW: 457, a yellow crystal, easily dissolved in a mixed solvent of chloroform and methanol.

¹H NMR (300 MHz, CDCl₃): δ 10.00 (1H, s, H-8), 7.84 (1H, d, J=9.0 Hz, H-2), 7.70 (1H, d, J=9.0 Hz, H-11), 7.13 (1H, s, H-1), 6.86 (1H, s, H-4), 6.0 (2H, s, —OCH₂O—), 4.28 (2H, m, H-3′), 4.26 (2H, m, H-6), 4.23 (3H, s, —OCH₃), 4.01 (3H, s, —OCH₃), 3.62 (2H, s, H-1′), 3.10 (2H, m, H-5), 1.28 (3H, t, J=7.2 Hz, H-4′).

Preparation of Compound 10

Dihydroberberine (337 mg) was dissolved in 35 mL of dichloromethane. Under argon atmosphere, the temperature of the system was kept at −25° C.˜−30° C., and 8 mL of dichloromethane solution containing 258 mg MCPBA (1.5 mmol) was dropwise added slowly. After the addition, the temperature was kept and the reaction was carried out under agitation for 1 hour. Then, the temperature was raised to 0° C. and 250 mg (2.0 mmol) of sodium sulfite was added therein, followed by stirred at room temperature for 1 hour. The reaction was stopped and the reaction mixture was filtered. The filtrate was evaporated to remove the solvent under reduced pressure and the residue was purified through silica gel column chromatography (CHCl₃/CH₃OH=10:1) to obtain compound 10 (280 mg, 80%).

Compound 10, C₂₀H₁₈NO₅, MW: 352, a yellow powder, easily dissolved in a mixed solvent of chloroform and methanol.

¹H NMR (300 MHz, CDCl₃), δ 8.83 (1H, s, H-8), 8.28 (1H, d, J=8.1 Hz, H-11), 7.78 (1H, s, H-1), 7.29 (1H, d, J=8.1 Hz, H-12), 6.52 (1H, s, H-4), 5.88 (2H, s, —OCH₂O—), 4.44 (2H, t, J=6.3 Hz, H-6), 3.95 (3H, s, OMe-9 or 10), 3.94 (3H, s, OMe-10 or 9), 2.95 (2H, t, J=6.3 Hz, H-5).

Preparation of Compound 11

Compound 10 (1.0 g) and potassium carbonate (200 mg) were dissolved in 20 mL of acetone followed by adding 0.2 mL of methyl iodide. The obtained reaction solution was heated and refluxed for 3 hours. Then, the reaction mixture was filtered and the filtrate was evaporated under reduced pressure. The residual was purified through silica gel column (CHCl₃/CH₃OH=20:1) to obtain compound 11 (0.41 g, 37%).

Compound 11, C₂₁H₂₀INO₅, MW: 493, a brown powder, easily dissolved in a mixed solvent of chloroform and methanol.

¹H NMR (300 MHz, CDCl₃), δ 8.97 (1H, s, H-8), 8.33 (1H, d, J=8.1 Hz, H-11), 7.91 (1H, s, H-1), 7.45 (1H, d, J=8.1 Hz, H-12), 6.76 (1H, s, H-4), 5.97 (2H, s, —OCH₂O—), 4.32 (2H, t, J=6.3 Hz, H-6), 4.01 (3H, s, —OMe), 3.94 (3H, s, —OMe), 3.73 (3H, s, —OMe), 2.95 (2H, t, J=6.3 Hz, H-5).

Preparation of Compound 12

Compound 10 (1.0 g) and potassium carbonate (200 mg) were dissolved in 20 mL of acetone followed by adding 0.25 mL of ethyl bromide. The obtained reaction solution was heated and refluxed for 3 hours. Then, the reaction mixture was filtered and the filtrate was evaporated under reduced pressure. The residual was purified through silica gel column (CHCl₃/CH₃OH=20:1) to obtain compound 12 (0.37 g, 34%).

Compound 12, C₂₂H₂₂BrNO₅, MW: 459, a brown yellow powder, easily dissolved in a mixed solvent of chloroform and methanol.

¹H NMR (300 MHz, CDCl₃), δ 9.01 (1H, s, H-8), 8.45 (1H, d, J=8.1 Hz, H-11), 7.99 (1H, s, H-1), 7.63 (1H, d, J=8.1 Hz, H-12), 6.81 (1H, s, H-4), 6.08 (2H, s, —OCH₂O—), 4.35 (2H, t, J=6.3 Hz, H-6), 4.11 (3H, s, —OMe), 4.01 (3H, s, —OMe), 3.92 (2H, m, H-1′), 2.95 (2H, t, J=6.3 Hz, H-5), 1.35 (3H, t, J=7.5 Hz, H-2′).

Preparation of Compound 13

Compound 10 (34 mg, 0.1 mmol) was dissolved in a mixed solution of 1 mL of pyridine and 1 mL of acetic anhydride. The mixture was allowed to react under agitation at room temperature overnight. Then, the reaction mixture was evaporated to remove the solvent under reduced pressure. The residual was purified through Sephdex LH-20 chromatography (CHCl₃/CH₃OH=10:1) to obtain compound 13 (28 mg, 80%).

Compound 13, C₂₂H₂₀NO₆, MW: 394, a yellow powder, easily dissolved in a mixed solvent of chloroform and methanol.

¹H NMR (300 MHz, CDCl₃), δ 8.81 (1H, s, H-8), 8.27 (1H, d, J=8.1 Hz, H-11), 7.76 (1H, s, H-1), 7.29 (1H, d, J=8.1 Hz, H-12), 6.56 (1H, s, H-4), 5.89 (2H, s, —OCH₂O—), 4.45 (2H, t, J=6.3 Hz, H-6), 3.95 (3H, s, OMe-9 or 10), 3.94 (3H, s, OMe-10 or 9), 2.95 (2H, t, J=6.3 Hz, H-5), 2.10 (3H, s, OAc).

Preparation of Compound 14

Berberine (4.5 g, 12.1 mmol) was added into a 50 mL round bottom flask. The reaction system was kept under a reduced pressure (20˜30 mmHg) using an oil pump, and heated to 190° C. followed by reacting for 40 minutes. The vacuum pump was switched off after the temperature dropped to room temperature. The reaction product was purified through silica gel column chromatography (CHCl₃/CH₃OH=15:1 and 10:1, eluting until no compound was observed in the eluent) to obtain compound 14 (3.3 g, 85%).

Compound 14, C₁₉H₁₅NO₄, MW: 321, a brownish red amorphous powder, easily dissolved in a mixed solvent of chloroform and methanol.

¹H NMR (300 MHz, CDCl₃), δ 9.06 (1H, s, H-8), 8.60 (1H, s, H-13), 8.18 (1H, d, J=8.1 Hz, H-11), 8.10 (1H, d, J=8.1 Hz, H-12), 7.57 (1H, s, H-1), 6.87 (1H, s, H-4), 6.10 (2H, s, —OCH₂O—), 4.90 (2H, t, J=6.3 Hz, H-6), 4.63 (3H, s, OMe-10), 3.20 (2H, t, J=6.3 Hz, H-5).

Preparation of Compound 15

Compound 14 (33 mg, 0.1 mmol) was dissolved in a mixed solution of 1 mL of pyridine and 1 mL of acetic anhydride. The obtained solution reacted under agitation at room temperature overnight. Then, the reaction mixture was evaporated to remove solvent under reduced pressure. The residual was purified through Sephdex LH-20 chromatography (CHCl₃/CH₃OH=10:1) to obtain compound 15 (32 mg, 88%).

Compound 15, C₂₁H₁₈NO₅, MW: 364, a red powder, easily dissolved in a mixed solvent of chloroform and methanol.

¹H NMR (300 MHz, CDCl₃+CD₃OD): δ 9.16 (1H, s, H-8), 8.64 (1H, s, H-13), 8.18 (1H, d, J=8.1 Hz, H-11), 8.04 (1H, d, J=8.1 Hz, H-12), 7.55 (1H, s, H-1), 6.87 (1H, s, H-4), 6.09 (2H, s, —OCH₂O—), 4.99 (2H, t, J=6.3 Hz, H-6), 4.03 (3H, s, OMe-10), 3.20 (2H, t, J=6.3 Hz, H-5), 2.10 (3H, s, OAc).

Preparation of Compound 16

Compound 14 (360 mg, 1.0 mmol) and triethylamine (1.08 g) were dissolved in 50 mL of anhydrous dichloromethane under agitation at room temperature. Then, 3 mL dichloromethane solution containing ethyl chlorformate (1.2 g, 11.1 mmol) was dropwise added slowly. After the addition, the reaction was performed for another 30 min. Then, the reaction mixture was evaporated to remove solvent under reduced pressure. The residual was purified through Sephdex LH-20 chromatography (CHCl₃/CH₃OH=1:1) to obtain compound 16 (202 mg, 88%).

Compound 16, C₂₂H₂₀NO₆, MW: 394, a yellow amorphous powder, easily dissolved in chloroform and methanol.

¹H NMR (300 MHz, CDCl₃): δ 9.18 (1H, s, H-8), 8.60 (1H, s, H-13), 8.12 (1H, d, J=8.1 Hz, H-11), 8.06 (1H, d, J=8.1 Hz, H-12), 7.54 (1H, s, H-1), 6.57 (1H, s, H-4), 6.04 (2H, s, —OCH₂O—), 4.49 (2H, t, J=6.3 Hz, H-6), 4.25 (2H, q, J=6.0 Hz, H-2′), 4.13 (3H, s, OMe-10), 3.20 (2H, t, J=6.3 Hz, H-5), 1.10 (3H, t, J=6.0 Hz, H-3′).

Preparation of Compound 17

Berberine (370 mg, 1.0 mmol) and 1,3,5-trihydroxybenzene (504 mg, 4.0 mmol) were dissolved in 10 mL of 60% sulphuric acid solution (volume ratio). Then, the obtained solution was heated to 80° C. and reacted under refluxing for 1 hour. The reaction mixture was cooled down, and 15 mL of sodium iodide solution (30 mg/mL) was added therein under ice bath. The reaction mixture was filtered, and the filter cake was dissolved in 2 mL of 1% potassium hydroxide solution. pH of the solution was adjusted to 4˜5 with sodium bisulfite. The mixture was filtered again, and the filter cake was purified by Sephdex LH-20 chromatography (CHCl₃/CH₃OH=10:1) to obtain compound 17 (108 mg, 33%).

Compound 17, C₁₉H₁₈ClNO₄, MW: 359, a yellow powder, easily dissolved in a mixed solvent of chloroform and methanol.

¹H NMR (300 MHz, DMSO), δ 9.83 (1H, s, H-8), 8.75 (1H, s, H-13), 8.18 (1H, d, J=8.1 Hz, H-11), 8.04 (1H, d, J=8.1 Hz, H-12), 7.53 (1H, s, H-1), 6.84 (1H, s, H-4), 4.89 (2H, t, J=6.3 Hz, H-6), 4.08 (3H, s, OMe-9 or 10), 4.05 (3H, s, OMe-10 or 9), 3.11 (2H, t, J=6.3 Hz, H-5).

Preparation of Compound 18

Compound 2 (490 mg, 1.0 mmol) and 1,3,5-trihydroxybenzene (504 mg, 4.0 mmol) were dissolved in 10 mL of 60% sulphuric acid solution (volume ratio). The obtained solution was heated to 80° C. and reacted under refluxing for 1 hour. The reaction mixture was cooled down, and 15 mL of sodium iodide solution (30 mg/mL) was added therein under ice bath. The reaction mixture was filtered, and the filter cake was dissolved in 2 mL of 1% potassium hydroxide solution. pH of the solution was adjusted to 4˜5 with sodium bisulfite. The mixture was filtered again, and the filter cake was purified through Sephdex LH-20 chromatography (CHCl₃/CH₃OH=10:1) to obtain compound 18 (123 mg, 31%).

Compound 18, C₂₁H₂₂ClNO₄, MW: 387, a yellow powder, easily dissolved in a mixed solvent of chloroform and methanol.

¹H NMR (300 MHz, DMSO-d₆), δ 9.96 (1H, s, H-8), 8.91 (1H, s, H-13), 8.23 (1H, d, J=8.1 Hz, H-11), 8.11 (1H, d, J=8.1 Hz, H-12), 7.72 (1H, s, H-1), 6.91 (1H, s, H-4), 4.98 (2H, t, J=6.3 Hz, H-6), 4.23 (3H, s, OMe-9 or 10), 4.15 (3H, s, OMe-10 or 9), 4.01 (2H, m, H-1′), 3.27 (2H, t, J=6.3 Hz, H-5), 1.45 (3H, t, J=7.5 Hz, H-2′).

Preparation of Compound 19

Berberine (370 mg, 1.0 mmol) was dissolved in 10 mL of anhydrous tetrahydrofuran followed by adding 190 mg of LiAlH₄ (5.0 mmol). The reaction was performed at room temperature under agitation for 2 hours. After the reaction finished, the reaction mixture was evaporated to remove the solvent under reduced pressure, and 0.2 mL of water, 0.2 mL of 30% sodium hydroxide solution and 0.6 mL of water were sequentially added therein. The reaction solution was then filtered, and the filtrate was extracted with ethyl acetate (10 mL×3). The obtained organic phase was dried over anhydrous sodium sulfate and evaporated under reduced pressure. The residual was purified through silica gel column chromatography (CHCl₃/CH₃OH=50:1, eluting until no compound was observed in the eluent) to obtain compound 19 (240 mg, 65%).

Compound 19, C₂₀H₁₉NO₄, MW: 337, a yellow amorphous powder, easily dissolved in chloroform and acetone.

¹H NMR (300 MHz, CDCl₃), δ 7.18 (1H, d, J=8.7 Hz, H-11), 6.73 (2H, m, H-12 and H-1), 6.56 (1H, s, H-4), 5.95 (1H, s, H-13), 5.94 (2H, s, —OCH₂O—), 4.32 (2H, s, H-8), 3.84 (6H, s, OMe×2), 3.20 (2H, t, J=8.1 Hz, H-6), 2.90 (2H, t, J=8.1 Hz, H-5).

Preparation of Compound 20

Magnesium strip (240 mg, 10 mmol) and ethyl bromide (1.08 g, 10 mmol) were dissolved in 15 mL of anhydrous diethyl ether under argon atmosphere. The reactant mixture was refluxed for another 2 hours after the violent reaction quieted down. The reaction solution was cooled down to 0° C., and berberine (370 mg, 1.0 mmol) was slowly added in batch. The ice bath was removed, and the reaction was carried out at room temperature overnight. Then, the reaction solution was injected into ice water (20 mL), and pH of the mixture was adjusted to 5 with a 2 N hydrochloric acid solution. The water phase was separated from the diethyl ether phase, cooled down, adjusted to have a pH of 11˜12 with a concentrated ammonia solution, and then extracted with chloroform (20 mL×3). The obtained organic phase was dried over anhydrous sodium sulfate, and evaporated under reduced pressure. The obtained oil was recrystallized in diethyl ether to obtain compound 20 (220 mg, 59%).

Compound 20, C₂₂H₂₃NO₄, MW: 365, a yellow powder crystal, easily dissolved in chloroform and acetone.

¹H NMR (300 MHz, CDCl₃): δ 7.12 (1H, s, H-1), 6.80 (2H, m, H-11 and 12), 6.57 (1H, s, H-4), 6.00 (2H, s, —OCH₂O—), 5.89 (1H, s, H-13), 3.85 (6H, s, OMe×2), 3.32 (2H, m, H-6), 3.06 (1H, t, J=6.0 Hz, H-8), 2.81 (2H, m, H-5), 1.72 (2H, m, H-1′), 0.94 (3H, t, J=6.3 Hz, H-2′).

Preparation of Compound 21

Magnesium strip (240 mg, 10 mmol) and benzyl bromide (1.7 g, 10 mmol) were dissolved in 15 mL of anhydrous diethyl ether under argon atmosphere. The reaction mixture was refluxed for another 2 hours after the violent reaction quieted down. The reaction solution was cooled to 0° C., and berberine (370 mg, 1.0 mmol) was slowly added in batch. The ice bath was removed, and the reaction was carried out at room temperature overnight. The reaction solution was injected into ice water (20 mL), and pH of the mixture was adjusted to 5 with a 2 mol/L hydrochloric acid solution. The water phase was separated from the diethyl ether phase, adjusted to have a pH of 11˜12 with a concentrated ammonia solution, and then extracted with chloroform (20 mL×3). The obtained organic phase was dried over anhydrous sodium sulfate and evaporated under reduced pressure. The obtained oil was recrystallized in diethyl ether to give compound 21 (280 mg, 59%).

Compound 21, C₂₇H₂₅NO₄, MW: 427, a yellow powder crystal, easily dissolved in chloroform and acetone. ¹H NMR (300 MHz, CDCl₃), δ 8.80 (1H, s, H-1), 8.53 (1H, d, J=8.7 Hz, H-11), 7.39 (1H, d, J=8.7 Hz, H-12), 7.08-7.34 (6H, m), 6.53 (1H, s, H-13), 6.00 (2H, s, —OCH₂O—), 4.35 (2H, t, J=6.0 Hz, H-6), 3.95 (3H, s, OMe), 3.93 (1H, m, H-8), 3.81 (1H, m, H-113), 3.43 (3H, s, OMe), 2.75 (3H, m, H-5 and 1′α); ESIMS m/z 428.2 ([M+H]⁺).

Preparation of Compound 22

Berberine (370 mg, 1.0 mmol) was dissolved in 10 mL of ethanol (analytical grade) under argon atmosphere, followed by adding 1.5 g of sodium ethylate. The reaction was carried out under agitation at room temperature overnight. The reaction mixture was evaporated to remove the solvent under reduced pressure, followed by adding 10 mL of water (which had been alkalified with sodium hydroxide). The mixture was then extracted with ethyl acetate (10 mL×3), and the obtained organic phase was dried over anhydrous sodium sulfate and evaporated under reduced pressure. The obtained yellow powder was recrystallized in methanol to give a needle like crystal 22 (94 mg, 27%).

Compound 22, C₂₀H₁₉NO₅, MW: 353, a yellow needle like crystal, easily dissolved in chloroform and acetone.

¹H NMR (300 MHz, CDCl₃): δ 7.16 (1H, s, H-1), 6.97 (1H, d, J=8.7 Hz, H-11), 6.88 (1H, d, J=8.7 Hz, H-12), 6.62 (1H, s, H-4), 6.10 (1H, s, H-13), 5.95 (2H, s, —OCH₂O—), 5.64 (1H, s, H-8), 3.88 (3H, s, OMe-9 or 10), 3.86 (3H, s, OMe-10 or 9), 3.86 (1H, m, H-6β), 3.72 (1H, m, H-6α), 3.36 (1H, m, H-5β), 2.76 (1H, m, H-5α).

Preparation of Compound 23

Berberine (370 mg, 1.0 mmol) was dissolved in 10 mL of methanol (analytical grade) under argon atmosphere, followed by adding 1.5 g of sodium methylate. The reaction was carried out under agitation at room temperature overnight. The reaction mixture was evaporated to remove the solvent under reduced pressure, followed by adding 10 mL of water (which had been alkalified with sodium hydroxide). The mixture was then extracted with ethyl acetate (10 mL×3), and the obtained organic phase was dried over anhydrous sodium sulfate and evaporated under reduced pressure. The obtained yellow powder was recrystallized in methanol to give a needle like crystal 23 (114 mg, 30%).

Compound 23, C₂₁H₂₁NO₅, MW: 367, a yellow needle like crystal, easily dissolved in chloroform and acetone.

¹H NMR (300 MHz, CDCl₃), δ 7.17 (1H, s, H-1), 6.96 (1H, d, J=8.7 Hz, H-11), 6.89 (1H, d, J=8.7 Hz, H-12), 6.64 (1H, s, H-4), 6.11 (1H, s, H-8), 6.03 (1H, s, H-13), 6.00 (2H, s, —OCH₂O—), 3.88 (3H, s, OMe-9 or 10), 3.86 (3H, s, OMe-10 or 9), 3.64 (1H, m, H-6α), 3.52 (1H, m, H-6β), 3.05 (3H, s, OMe-8), 2.89 (2H, m, H-5).

Preparation of Compound 24

Metallic sodium (0.85 g) was slowly added into 10 mL of anhydrous ethanol. After the addition, berberine 370 mg (1.0 mmol) was added under argon atmosphere. The reaction was carried out under agitation at room temperature overnight. Then, the reaction mixture was evaporated to remove the solvent under reduced pressure, followed by adding 10 mL of water (which had been alkalified with sodium hydroxide). The mixture was extracted with ethyl acetate (10 mL×3), and the obtained organic phase was dried over anhydrous sodium sulfate and evaporated under reduced pressure. The obtained yellow powder was recrystallized in ethanol to give a yellow needle like crystal 24 (130 mg, 34%).

Compound 24, C₂₂H₂₃NO₅, MW: 381, a yellow needle like crystal, easily dissolved in chloroform and acetone.

¹H NMR (300 MHz, CDCl₃), δ 7.17 (1H, s, H-1), 6.95 (1H, d, J=8.7 Hz, H-11), 6.86 (1H, d, J=8.7 Hz, H-12), 6.63 (1H, s, H-4), 6.13 (1H, s, H-8), 6.03 (1H, s, H-13), 6.01 (2H, s, —OCH₂O—), 3.88 (3H, s, OMe-9 or 10), 3.85 (3H, s, OMe-10 or 9), 3.60 (1H, m, H-6α), 3.51 (1H, m, H-6β), 3.25 (2H, q, J=6.3 Hz, H-1′), 2.87 (2H, m, H-5), 1.00 (3H, t, J=6.3 Hz, H-2′).

Preparation of Compound 25

Berberine (370 mg, 1.0 mmol) was dissolved in 10 mL of chloroform (analytical grade) under argon atmosphere, followed by adding 0.5 g of sodium hydride. The reaction was carried out under agitation at room temperature overnight. Then, the reaction mixture was evaporated to remove the solvent under reduced pressure, followed by adding 10 mL of water (which had been alkalified with sodium hydroxide). The mixture was then extracted with ethyl acetate (10 mL×3), and the obtained organic phase was dried over anhydrous sodium sulfate and evaporated under reduced pressure. The obtained yellow powder was recrystallized in methanol to give a yellow needle like crystal 25 (80 mg, 21%).

Compound 25, C₂₁H₁₈Cl₃NO₄, MW: 453, a yellow needle like crystal, easily dissolved in chloroform and acetone.

¹H NMR (300 MHz, CDCl₃), δ 7.16 (1H, s, H-1), 6.97 (1H, d, J=8.7 Hz, H-11), 6.86 (1H, d, J=8.7 Hz, H-12), 6.61 (1H, s, H-4), 6.10 (1H, s, H-13), 6.00 (2H, s, —OCH₂O—), 5.64 (1H, s, H-8), 3.94 (3H, s, OMe-9 or 10), 3.88 (3H, s, OMe-10 or 9), 3.87 (1H, m, H-6α), 3.70 (1H, m, H-6β), 2.89 (2H, m, H-5).

Preparation of Compound 26

Berberine (1 g) was dissolved in a 5 mol/L NaOH aqueous solution (5 mL), and acetone (2 mL) was dropwise added therein under agitation. The reaction was performed at room temperature for another 1 hour. The reaction mixture was filtered, and the filter cake was washed with methanol to give a yellow solid powder 26 (780 mg, 78%).

Compound 26, C₂₃H₂₃NO₅, MW: 393, a yellow amorphous powder, easily dissolved in chloroform and acetone.

¹H NMR (300 MHz, CDCl₃): δ 7.13 (1H, s, H-1), 6.78 (2H, m, H-11 and 12), 6.57 (1H, s, H-4), 5.94 (2H, s, —OCH₂O—), 5.89 (1H, s, H-13), 5.32 (1H, dd, J=6.9, 15.3 Hz, H-8), 3.85 (6H, s, OMe×2), 3.32 (2H, m, H-6), 3.06 (1H, dd, J=3.9, 6.9 Hz, H-1′α), 2.81 (2H, m, H-5), 2.42 (1H, dd, J=3.9, 15.3 Hz, H-1′β), 2.04 (3H, s, H-3′).

Preparation of Compound 27

Compound 2 (480 mg) was dissolved in 20 mL of anhydrous tetrahydrofuran followed by adding LiAlH₄ (120 mg) slowly. The reaction was carried out at room temperature for 3 hours. Water (0.1 mL), 5N sodium hydroxide solution (0.1 mL) and water (0.9 mL) were dropwise added into the reaction solution sequentially. Then, the reaction mixture was filtered, and the filtrate was evaporated to dryness under reduced pressure. The residue was recrystallized in dichloromethane-methanol mixed solvent to give compound 27 (300 mg, 61%).

Compound 27, C₂₁H₂₁NO₄, MW: 351, a yellow powder crystal, easily dissolved in chloroform and acetone.

¹H NMR (300 MHz, CDCl₃): δ 7.17 (1H, s, H-1), 7.03 (1H, d, J=8.4 Hz, H-11), 6.84 (1H, d, J=8.4 Hz, H-12), 6.68 (1H, s, H-4), 5.98 (2H, s, —OCH₂O—), 4.33 (2H, s, H-8), 3.86 (6H, s, —OCH₃), 3.13 (2H, m, H-6), 2.83 (2H, m, H-5), 2.78 (3H, s, —CH₃).

Preparation of Compound 28

Compound 3 (490 mg) was dissolved in 20 mL of anhydrous tetrahydrofuran followed by adding LiAlH₄ (120 mg) slowly. The reaction was carried out at room temperature for 3 hours. Water (0.1 mL), 5N sodium hydroxide solution (0.1 mL) and water (0.9 mL) were dropwise added into the reaction solution sequentially. Then, the reaction mixture was filtered, and the filtrate was evaporated to dryness under reduced pressure. The residue was recrystallized in dichloromethane-methanol mixed solvent to give compound 28 (320 mg, 63%).

Compound 28, C₂₂H₂₃NO₄, MW: 365, a yellow powder crystal, easily dissolved in chloroform and acetone.

¹H NMR (300 MHz, CDCl₃): δ 7.19 (1H, s, H-1), 7.09 (1H, d, J=8.4 Hz, H-11), 6.77 (1H, d, J=8.4 Hz, H-12), 6.69 (1H, s, H-4), 6.01 (2H, s, —OCH₂O—), 4.23 (2H, s, H-8), 3.78 (6H, s, —OCH₃), 3.13 (2H, m, H-6), 2.83 (2H, m, H-5), 2.78 (2H, m, H-1′), 1.34 (3H, t, J=7.5 Hz, H-2′).

Preparation of Compound 29

Magnesium strip (240 mg, 10 mmol) and methyl iodide (1.40 g, 10 mmol) were dissolved in 15 mL of anhydrous diethyl ether under argon atmosphere. The reaction mixture was refluxed for another 2 hours after the violent reaction quieted down. The reaction solution was cooled down to 0° C., and berberine (370 mg, 1.0 mmol) was slowly added in batch. The ice bath was removed, and the reaction was carried out at room temperature overnight. Then, the reaction solution was injected into ice water (20 mL), and pH of the mixture was adjusted to 5 with a 2 N hydrochloric acid solution. The water phase was separated from the diethyl ether phase, cooled down, adjusted to have a pH of 11˜12 with a concentrated ammonia solution, and then extracted with chloroform (20 mL×3). The obtained organic phase was dried over anhydrous sodium sulfate, and evaporated under reduced pressure. The obtained oil was recrystallized in diethyl ether to give compound 29 (245 mg, 61%).

Compound 29, C₂₁H₂₁NO₄, MW: 351, a yellow powder crystal, easily dissolved in chloroform and acetone.

¹H NMR (300 MHz, CDCl₃): δ 7.17 (1H, s, H-1), 6.85 (2H, m, H-11 and 12), 6.62 (1H, s, H-4), 6.05 (2H, s, —OCH₂O—), 5.94 (1H, s, H-13), 3.90 (6H, s, OMe×2), 3.37 (2H, m, H-6), 3.11 (1H, t, J=6.0 Hz, H-8), 2.86 (2H, m, H-5), 1.31 (3H, t, J=7.0 Hz, H-2′).

Preparation of Compound 30

Magnesium strip (240 mg, 10 mmol) and methyl iodide (1.40 g, 10 mmol) were dissolved in 15 mL of anhydrous diethyl ether under argon atmosphere. The reaction mixture was refluxed for another 2 hours after violent reaction quieted down. The reaction solution was cooled down to 0° C., and compound 2 (480 mg, 1.0 mmol) was slowly added in batch. The ice bath was removed, and the reaction was carried out at room temperature overnight. Then, the reaction solution was injected into ice water (20 mL), and pH of the mixture was adjusted to 5 with a 2 N hydrochloric acid solution. The water phase was separated from the diethyl ether phase, cooled down, adjusted to have a pH of 11˜12 with a concentrated ammonia solution, and then extracted with chloroform (20 mL×3). The obtained organic phase was dried over anhydrous sodium sulfate, and evaporated under reduced pressure. The obtained oil was recrystallized in diethyl ether to give compound 30 (232 mg, 43%).

Compound 30, C₂₂H₂₃NO₄, MW: 365, a yellow powder crystal, easily dissolved in chloroform and acetone.

¹H NMR (300 MHz, CDCl₃): δ 7.09 (1H, s, H-1), 6.81 (2H, m, H-11 and 12), 6.52 (1H, s, H-4), 6.01 (2H, s, —OCH₂O—), 3.73 (6H, s, OMe×2), 3.21 (2H, m, H-6), 3.08 (1H, m, H-8), 2.86 (2H, m, H-5), 1.83 (3H, s, H-1′), 1.38 (3H, d, J=6.3 Hz, H-2′).

Preparation of Compound 31

Magnesium strip (240 mg, 10 mmol) and ethyl bromide (1.08 g, 10 mmol) were dissolved in 15 mL of anhydrous diethyl ether under argon atmosphere. The reaction mixture was refluxed for another 2 hours after the violent reaction quieted down. The reaction solution was cooled down to 0° C., and compound 3 (490 mg, 1.0 mmol) was slowly added in batch. The ice bath was removed, and the reaction was carried out at room temperature overnight. Then, the reaction solution was injected into ice water (20 mL), and pH of the mixture was adjusted to 5 with a 2 N hydrochloric acid solution. The water phase was separated from the diethyl ether phase, cooled down, adjusted to have a pH of 11˜12 with a concentrated ammonia solution, and then extracted with chloroform (20 mL×3). The obtained organic phase was dried over anhydrous sodium sulfate, and evaporated under reduced pressure. The obtained oil was recrystallized in diethyl ether to give compound 31 (220 mg, 43%).

Compound 31, C₂₄H₂₇NO₄, MW: 393, a yellow powder crystal, easily dissolved in chloroform and acetone.

¹H NMR (300 MHz, CDCl₃): δ 7.07 (1H, s, H-1), 6.75 (2H, m, H-11 and 12), 6.51 (1H, s, H-4), 5.98 (2H, s, —OCH₂O—), 3.85 (6H, s, OMe×2), 3.32 (2H, m, H-6), 3.06 (1H, t, J=6.0 Hz, H-8), 2.98 (2H, m, H-3′), 2.81 (2H, m, H-5), 1.72 (2H, m, H-1′), 1.33 (3H, t, J=6.0 Hz, H-4′), 0.94 (3H, t, J=6.3 Hz, H-2′).

Preparation of Compound 32

Compound 19 (337 mg, 1.0 mmol) and 1,3,5-trihydroxybenzene (504 mg, 4.0 mmol) were dissolved in 10 mL of 60% sulphuric acid solution (volume ratio). The obtained solution was then heated to 80° C., and reacted under refluxing for 1 hour. The reaction mixture was cooled down and extracted with ethyl acetate. The obtained organic phase was distilled to dryness using a rotary evaporator, and the residue was purified through a silica gel column (CHCl₃:MeOH=10:1) to obtain compound 32 (108 mg, 33%).

Compound 32, C₁₉H₁₉NO₄, MW: 325, a brown powder, easily dissolved in chloroform and methanol.

¹H NMR (300 MHz, CDCl₃): δ 6.87 (1H, s, H-1), 6.52 (1H, s, H-4), 6.75 (1H, d, J=8.1 Hz, H-11), 6.47 (1H, d, J=8.1 Hz, H-12), 5.88 (1H, s, H-13), 4.27 (2H, s, H-8), 3.76 (6H, s, —OCH₃), 3.01 (2H, t, J=6.3 Hz, H-6), 2.54 (2H, t, J=6.3 Hz, H-5).

Preparation of Compound 33

Compound 14 (321 mg, 1.0 mmol) and 1,3,5-trihydroxybenzene (504 mg, 4.0 mmol) were dissolved in 10 mL of 60% sulphuric acid solution (volume ratio). The obtained mixture was heated to 80° C., and reacted under refluxing for 1 hour. The reaction mixture was cooled down and extracted with ethyl acetate. The obtained organic phase was distilled to dryness using a rotary evaporator, and the residue was purified through a silica gel column (CHCl₃:MeOH=10:1) to obtain compound 33 (98 mg, 31%).

Compound 33, C₁₈H₁₇NO₄, MW: 311, a brown powder, easily dissolved in chloroform and methanol.

¹H NMR (300 MHz, CDCl₃): δ 6.92 (1H, s, H-1), 6.57 (1H, s, H-4), 6.70 (1H, d, J=8.0 Hz, H-11), 6.41 (1H, d, J=8.0 Hz, H-12), 5.83 (1H, s, H-13), 4.33 (2H, s, H-8), 3.87 (3H, s, —OCH₃), 3.11 (2H, t, J=6.3 Hz, H-6), 2.33 (2H, t, J=6.3 Hz, H-5).

Preparation of Compound 34

Compound 29 (351 mg, 1.0 mmol) and 1,3,5-trihydroxybenzene (504 mg, 4.0 mmol) were dissolved in 10 mL of 60% sulphuric acid solution (volume ratio). The obtained mixture was heated to 80° C., and reacted under refluxing for 1 hour. The reaction mixture was then cooled down and extracted with ethyl acetate. The obtained organic phase was distilled to dryness using a rotary evaporator, and the residue was purified through a silica gel column (CHCl₃:MeOH=10:1) to obtain compound 34 (103 mg, 32%).

Compound 34, C₂₀H₂₁NO₄, MW: 339, a brown powder, easily dissolved in chloroform and methanol.

¹H NMR (300 MHz, CDCl₃): δ 6.78 (1H, s, H-1), 6.47 (1H, s, H-4), 6.31 (1H, d, J=8.0 Hz, H-11), 6.23 (1H, d, J=8.0 Hz, H-12), 4.37 (2H, s, H-8), 3.73 (6H, s, —OCH₃), 3.11 (2H, t, J=6.3 Hz, H-6), 2.43 (2H, t, J=6.3 Hz, H-5), 1.79 (3H, s, —CH₃).

Preparation of Compound 35

Compound 27 (351 mg, 1.0 mmol) and 1,3,5-trihydroxybenzene (504 mg, 4.0 mmol) were dissolved in 10 mL of 60% sulphuric acid solution (volume ratio). The obtained mixture was heated to 80° C., and reacted under refluxing for 1 hour. The reaction mixture was then cooled down and extracted with ethyl acetate. The obtained organic phase was distilled to dryness using a rotary evaporator, and the residue was purified through a silica gel column (CHCl₃:MeOH=10:1) to obtain compound 35 (107 mg, 33%).

Compound 35, C₂₀H₂₁NO₄, MW: 339, a brown yellow powder, easily dissolved in chloroform and methanol.

¹H NMR (300 MHz, CDCl₃): δ 6.77 (1H, s, H-1), 6.45 (1H, s, H-4), 6.29 (1H, d, J=8.0 Hz, H-11), 6.21 (1H, d, J=8.0 Hz, H-12), 4.51 (2H, s, H-8), 3.71 (6H, s, —OCH₃), 3.13 (2H, t, J=6.3 Hz, H-6), 2.41 (2H, t, J=6.3 Hz, H-5), 1.34 (3H, s, —CH₃).

Preparation of Compound 36

Compound 30 (365 mg, 1.0 mmol) and 1,3,5-trihydroxybenzene (504 mg, 4.0 mmol) were dissolved in 10 mL of 60% sulphuric acid solution (volume ratio). The obtained mixture was heated to 80° C., and reacted under refluxing for 1 hour. The reaction mixture was then cooled down and extracted with ethyl acetate. The obtained organic phase was distilled to dryness using a rotary evaporator, and the residue was purified through a silica gel column (CHCl₃:MeOH=10:1) to obtain compound 36 (123 mg, 37%).

Compound 36, C₂₁H₂₃NO₄, MW: 353, a brown yellow powder, easily dissolved in chloroform and methanol.

¹H NMR (300 MHz, CDCl₃): δ 6.81 (1H, s, H-1), 6.55 (1H, s, H-4), 6.32 (1H, d, J=8.0 Hz, H-11), 6.22 (1H, d, J=8.0 Hz, H-12), 4.43 (2H, s, H-8), 3.70 (6H, s, —OCH₃), 3.34 (2H, t, J=6.3 Hz, H-6), 2.58 (2H, t, J=6.3 Hz, H-5), 1.79 (3H, s, —CH₃), 1.35 (3H, s, —CH₃).

Preparation of Compound 37

Compound 32 (325 mg) was dissolved in a mixture solution of 1 mL of pyridine and 1 mL of acetic anhydride. The reaction was carried out under agitation at room temperature overnight. The reaction mixture was then evaporated to remove the solvent under reduced pressure. The residue was purified through silica gel column chromatography (CHCl₃/CH₃OH=20:1) to obtain compound 37 (132 mg, 37%).

Compound 37, C₂₃H₂₃NO₆, MW: 409, a brown yellow powder, easily dissolved in chloroform and methanol.

¹H NMR (300 MHz, CDCl₃): δ 7.15 (1H, s, H-1), 6.77 (1H, d, J=8.1 Hz, H-11), 6.52 (1H, s, H-4), 6.47 (1H, d, J=8.1 Hz, H-12), 5.93 (1H, s, H-13), 4.42 (2H, s, H-8), 3.84 (6H, s, —OCH₃), 3.13 (2H, t, J=6.3 Hz, H-6), 2.87 (2H, t, J=6.3 Hz, H-5), 2.08 (6H, s, —OCOCH₃).

Preparation of Compound 38

2,3,9,10-tetrahydroxy dihydropalmatine (297 mg) was dissolved in a mixture solution of 1 mL of pyridine and 1 mL of acetic anhydride. The reaction was carried out under agitation at room temperature overnight. The reaction mixture was then neutralized with a saturated sodium bicarbonate solution and extracted with ethyl acetate. The obtained organic phase was evaporated under reduced pressure, and the residue was purified through silica gel column chromatography (CHCl₃/CH₃OH=25:1) to obtain compound 38 (108 mg, 31%).

Compound 38, C₂₃H₂₃NO₆, MW: 409, a brown yellow powder, easily dissolved in chloroform and methanol.

¹H NMR (300 MHz, CDCl₃): δ 7.21 (1H, s, H-1), 6.79 (1H, d, J=8.1 Hz, H-11), 6.55 (1H, s, H-4), 6.47 (1H, d, J=8.1 Hz, H-12), 5.91 (1H, s, H-13), 4.51 (2H, s, H-8), 3.17 (2H, t, J=6.3 Hz, H-6), 2.84 (2H, t, J=6.3 Hz, H-5), 2.08 (6H, s, —OCOCH₃), 2.06 (6H, s, —OCOCH₃).

Preparation of Compound 39

Palmatine hydrochloride (390 mg, 1.0 mmol) was dissolved in 10 mL anhydrous tetrahydrofuran followed by adding 190 mg of LiAlH₄ (5.0 mmol). The reaction was carried out under agitation at room temperature for 2 hours. After the reaction finished, the reaction mixture was evaporated to remove the solvent under reduced pressure, and 0.2 mL of water, 0.2 mL of 30% sodium hydroxide solution and another 0.6 mL of water were sequentially added therein. The reaction solution was then filtered, and the filtrate was extracted with ethyl acetate (10 mL×3). The obtained organic phase was dried over anhydrous sodium sulfate and evaporated under reduced pressure. The residue was purified through silica gel column chromatography (CHCl₃/CH₃OH=50:1, eluting until no compound was observed in the eluent) to obtain compound 39 (253 mg, 67%).

Compound 39, C₂₁H₂₃NO₄, MW: 353, a yellow amorphous powder, easily dissolved in chloroform and acetone.

¹H NMR (300 MHz, CDCl₃), δ 7.21 (1H, s, H-1), 7.08 (1H, d, J=8.4 Hz, H-12), 6.91 (1H, d, J=8.4 Hz, H-11), 6.67 (1H, s, H-4), 5.76 (1H, s, H-13), 4.33 (2H, s, H-8), 3.90 (6H, s, —OMe x 2), 3.86 (3H, s, —OMe), 3.83 (3H, s, —OMe), 3.18 (2H, t, J=7.5 Hz, H-6), 2.91 (2H, t, J=7.5 Hz, H-5).

Preparation of Compound 40

Palmatine hydrochloride (415 mg, 1.0 mmol) was dissolved in 10 mL anhydrous tetrahydrofuran followed by adding 190 mg of LiAlH₄ (5.0 mmol). The reaction was carried out under agitation at room temperature for 2 hours. After the reaction finished, the reaction mixture was evaporated to remove the solvent under reduced pressure, and 0.2 mL of water, 0.2 mL of 30% sodium hydroxide solution and another 0.6 mL of water were sequentially added therein. The reaction solution was then filtered, and the filtrate was extracted with ethyl acetate (10 mL×3). The obtained organic phase was dried over anhydrous sodium sulfate and evaporated under reduced pressure. The residue was purified through silica gel column chromatography (CHCl₃/CH₃OH=50:1, eluting until no compound was observed in the eluent) to obtain compound 40 (223 mg, 61%).

Compound 40, C₂₃H₂₇NO₄, MW: 381, a yellow amorphous powder, easily dissolved in chloroform and acetone.

¹H NMR (300 MHz, CDCl₃), δ 7.17 (1H, s, H-1), 7.03 (1H, d, J=8.4 Hz, H-12), 6.84 (1H, d, J=8.4 Hz, H-11), 6.68 (1H, s, H-4), 4.27 (2H, s, H-8), 3.92 (3H, s, —OMe), 3.91 (3H, s, —OMe), 3.88 (3H, s, —OMe), 3.85 (3H, s, —OMe), 3.08 (2H, m, H-6), 2.81 (4H, m, H-5 and 1′), 1.34 (3H, t, J=7.5 Hz, H-2′).

Preparation of Compound 41

Coptisine hydrochloride (355 mg, 1.0 mmol) was dissolved in 10 mL anhydrous tetrahydrofuran followed by adding 190 mg of LiAlH₄ (5.0 mmol). The reaction was carried out under agitation at room temperature for 2 hours. After reaction finished, the reaction mixture was evaporated to remove the solvent under reduced pressure, and 0.2 mL of water, 0.2 mL of 30% sodium hydroxide solution and another 0.6 mL of water were sequentially added therein. The reaction solution was then filtered, and the filtrate was extracted with ethyl acetate (10 mL×3). The obtained organic phase was dried over anhydrous sodium sulfate and evaporated under reduced pressure. The residue was purified through silica gel column chromatography (CHCl₃/CH₃OH=50:1, eluting until no compound was observed in the eluent) to obtain compound 41 (223 mg, 61%).

Compound 41, C₁₉H₁₅NO₄, MW: 321, a yellow amorphous powder, easily dissolved in chloroform and acetone.

¹H NMR (300 MHz, CDCl₃), δ 7.13 (1H, s, H-1), 7.02 (1H, d, J=8.4 Hz, H-12), 6.83 (1H, d, J=8.4 Hz, H-11), 6.64 (1H, s, H-4), 5.98 (2H, s, —OCH₂O—), 5.96 (2H, s, —OCH₂O—), 4.23 (2H, s, H-8), 3.11 (2H, t, J=7.5 Hz, H-6), 2.81 (2H, t, J=7.5 Hz, H-5).

### Experimental Examples

### Experimental Example 1

The effects of some compounds according to the present invention on glucose absorption were preliminarily evaluated in vitro using the glucose uptake model in L6 muscle cells.

Experimental Steps:

The completely differentiated L6 muscle cells which were cultured in 24-well plate were washed with PBS one time, starved in a high glucose DMEM culture medium containing 0.2% of BSA for 2 hours, and incubated in a high glucose DMEM culture medium containing 5 μM of the dihydroberberine derivatives of the present invention and 0.2% of BSA for 2.5 hours. They were then washed twice with a HBS solution containing 5 μM of the dihydroberberine derivatives, and incubated for another 0.5 hour in a HBS solution containing 5 μM of the dihydroberberine derivatives.

³H-labeled 2-deoxyglucose (dissolved in HBS or KRP to form a 1 mM, 5 μCi/mL temporary working solution) was added into a HBS solution to obtain a solution with a final concentration of 100 μM and 0.5 μCi/mL of the isotope. The cells were incubated at 37° C. for 10 min. The cell incubation solution was then removed quickly, and the cells were placed on ice, rinsed with ice-cold PBS three times, and dried in an oven at 42° C. 200 μL of 0.1% TritonX-100 was added therein, and the mixture was mildly vibrated at 4° C. for 45 min to lyse the cells. 150 μL of lysate was taken, and 1.1 mL of scintillation fluid was added therein to perform scinticounting. 10 μL of lysate was diluted by 10 times to measure the protein concentration using Bradford method. Final result is presented with a unit of pmol/min/mg protein.

Evaluation Standard:

The compound to be measured was dissolved in DMSO. When the concentration is 5 μM, if the calculated glucose intake in the test using the compound of the present invention is higher than that in DMSO blank control and there is significant difference in statistics between the data of the two groups, it can be concluded that the compound of the present invention can promote glucose absorption.

Experimental Result:

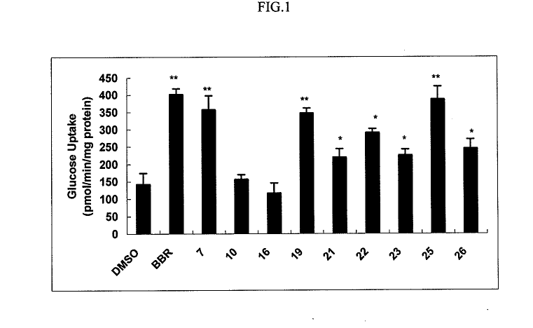

FIG. 1 shows the effects of some compounds of the present invention at 5 μM on promoting glucose transport in 5.0 mM glucose medium in glucose intake model in L6 muscle cells, wherein DMSO is blank control, and BBR is berberine.

It has been found, by screening at cellular level, that several compounds have stronger activity of promoting glucose uptake than berberine. Depending on the difficult level of synthesis, we selected dihydroberberine derivative 19 and its sulfate salt to perform the pharmacodynamical test at the whole animal level.

### Experiment Example 2

The in vivo antidiabetic activity of the compounds according to the present invention is evaluated.

Experimental Steps:

Normal male C57BL/6J mice were fed by high-fat diet for 10 weeks to generate obvious symptom of insulin resistance, and their ability of glucose tolerance decreased significantly. Ten mice per group were used to perform the efficacy study of the compounds. Dihydroberberine derivative 19 or its sulfate salt was mixed into the high-fat diet, and administered at a dose of 100 mg/kg/day for 2 weeks. After the mice were starved overnight, basal blood sugar value (0 min) was measured by taking blood from tail vein. Then the mice were injected glucose intraperitoneally at a dose of 2 g/kg according to the body weight of the mice in control group which were fed by normal diet followed by measuring blood sugar value at 15, 30, 45, 60, 90 and 120 min respectively and calculating the area under curve (AUC). The body weight and visceral fat of the mice were weighted, and the contents of fatty acid and triglyceride in the blood plasma of the mice were determined.

Experimental Result:

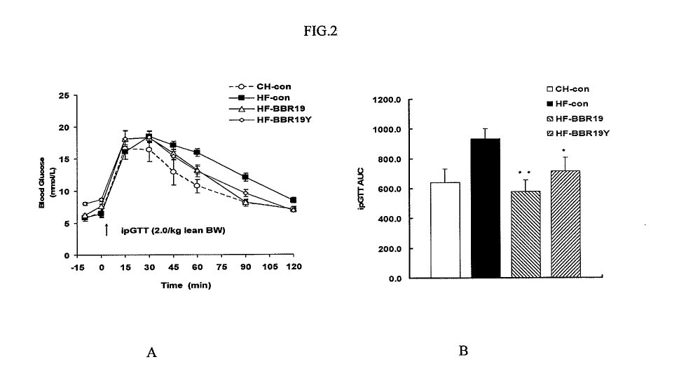

In FIGS. 2-4, CH-con means the normal mouse, HF-con refers to the obese mouse, HF-BBR19 is the obese mouse treated with dihydroberberine derivative 19 for 2 weeks, and HF-BBR19Y is the obese mouse treated with the sulfate salt of dihydroberberine derivative 19 for 2 weeks.

FIG. 2 shows the intraperitoneal glucose tolerance test (ipGTT) curves of the mice and the areas under curve. The mice which had been treated with dihydroberberine derivative 19 or its sulfate salt for 2 weeks and the mice in control group were intraperitoneally injected glucose at 2 g/kg after being starved overnight. The blood glucose value was then measured at 0, 15, 30, 45, 60, 90 and 120 min respectively and the area under curve (AUC) was calculated (*, P<0.05; **, P<0.01). It can be seen from FIG. 2 that the obese mice have a significantly improved glucose tolerance after being treated with dihydroberberine derivative 19 or its sulfate salt for 2 weeks.

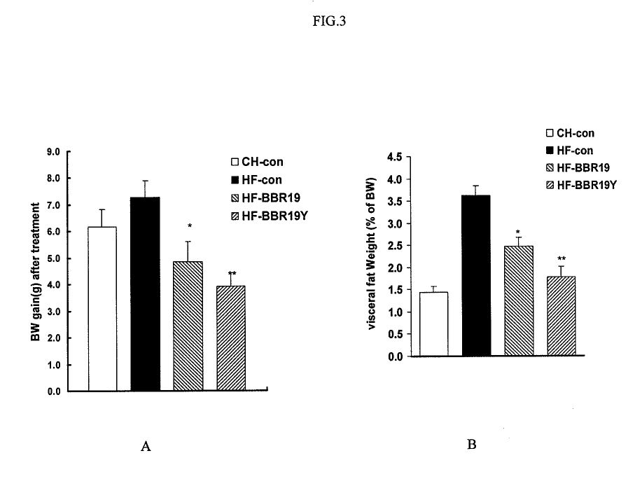

FIG. 3 shows the increase in body weight of the obese mice and their visceral fat/body weight ratio after treatment with dihydroberberine derivative 19 or its sulfate salt for 2 weeks (*, P<0.05; **, P<0.01). It can be seen from FIG. 3 that the increase in body weight of the mice which had been treated with dihydroberberine derivative 19 or its sulfate salt for 2 weeks is significantly less than that of the mice in the control group, and the ratio of intra-abdominal fat to body weight is lowered significantly comparing with the control group. Such results indicate that the compounds of the present invention have the effects on preventing the increase in body weight of the mice and fat accumulation induced by high-fat diet. Thus they may have a potential effect on treating obesity.

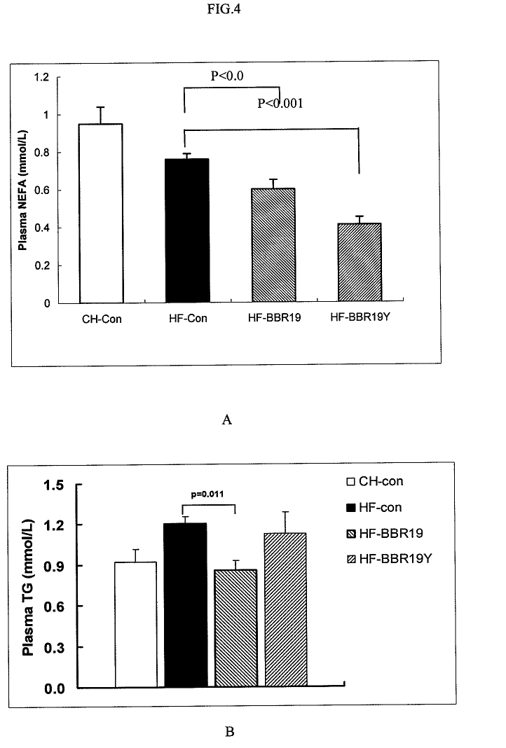

FIG. 4 shows the changes in the contents of free fatty acids (i.e. non-esterified fatty acid, NEFA) and triglyceride (TG) in the blood plasma of obese mice which have been treated with dihydroberberine derivative 19 or its sulfate salt for 2 weeks (*, P<0.05; **, P<0.01). It can be seen from FIG. 4 that the contents of free fatty acid (NEFA) (HF-Con vs HF-BBR19, 0.76±0.03 mmol/l vs 0.6±0.05 mmol/l) and triglyceride TG (HF-Con vs HF-BBR19, 1.20±0.05 mmol/l vs 0.86±0.08 mmol/l) in blood plasma of obese mice are both lowered significantly after the mice have been treated with dihydroberberine derivative 19 or sulfate thereof for 2 weeks.

### EXPERIMENTAL CONCLUSION

Dihydroberberine derivative 19 and its sulfate salt have significant effects on improving glucose tolerance and insulin resistance, facilitating weight loss, lowering the contents of free fatty acids and triglyceride in blood plasma and relieving fatty liver in the mouse models with insulin resistance and obesity induced by a high-fat diet.

1. A method of treating diabetes mellitus in a patient comprising administering to the patient an effective amount of a 13,13a-dihydroberberine derivative or its physiologically acceptable salt represented by Formula 1: 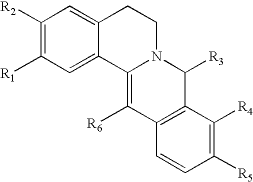
Wherein,
R₁ and R₂ are each independently H, OH, C₁-C₄ alkoxyl or C₁-C₄ acyloxy, or R₁ and R₂ are combined into —O—CH₂—O—;
R₃ is H, OH, C₁-C₄ alkoxyl, C₁-C₄ acyloxy, C₁-C₄ alkyl, C₁-C₄ haloalkyl or an aryl;
R₄ and R₅ are each independently H, OH, C₁-C₄ alkoxyl or C₁-C₄ acyloxy, or R₄ and R₅ are combined into —O—CH₂—O—; and
R₆ is H, OH, C₁-C₄ alkoxyl, C₁-C₄ acyloxy, C₁-C₄ alkyl, C₁-C₄ haloalkyl or an aryl.

2. The method according to claim 1, wherein the compound of Formula 1 is one selected from the following compounds represented by Formulae 19 to 41:

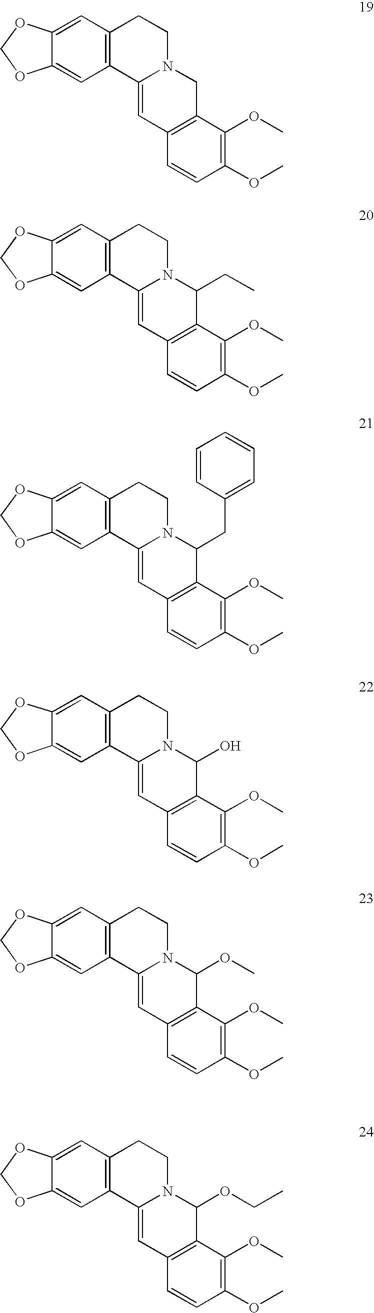

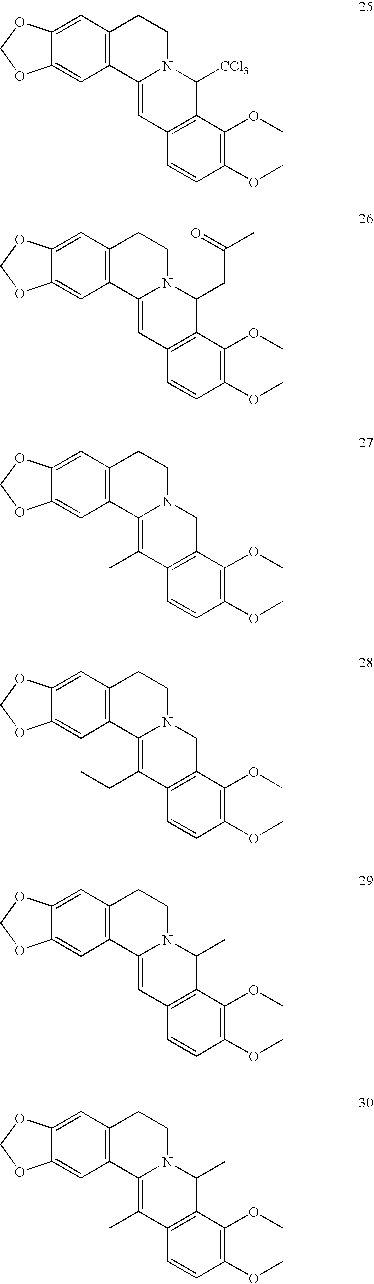

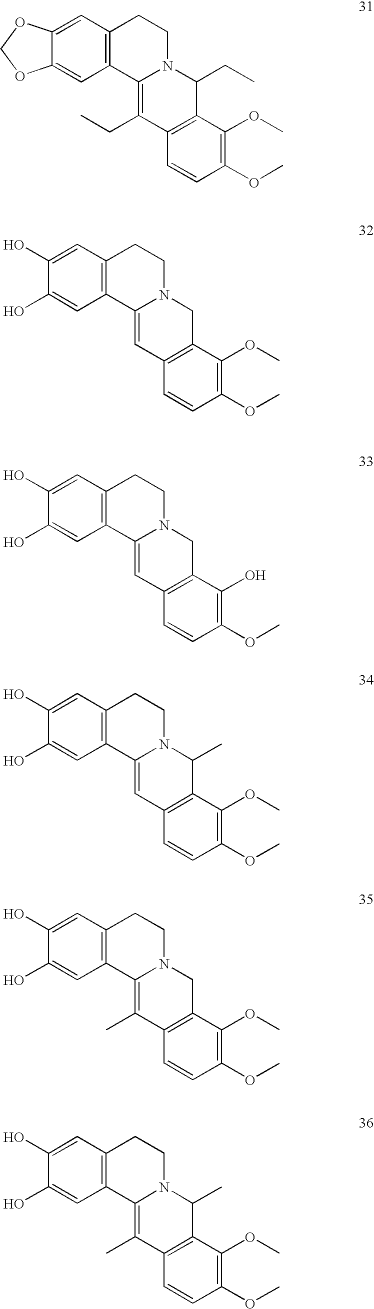

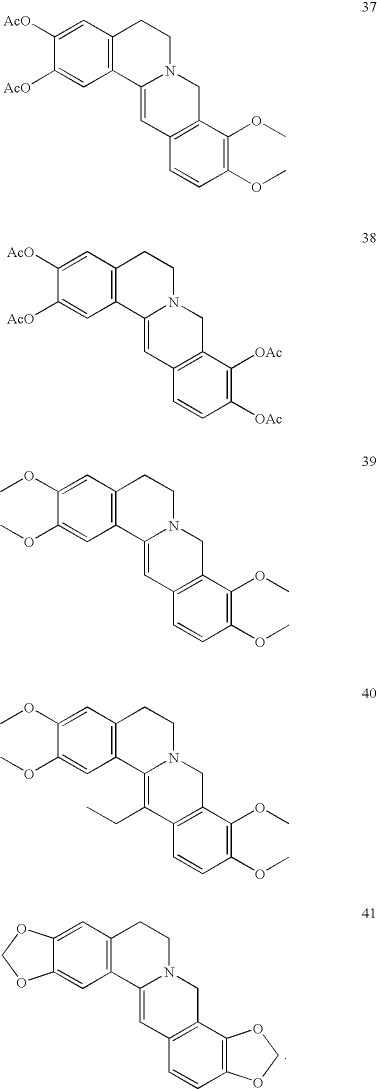

3. A method of treating a patient suffering from insulin resistance and complications thereof comprising administering to the patient an effective amount of a 13,13a-dihydroberberine derivative or its physiologically acceptable salt represented by Formula 1: 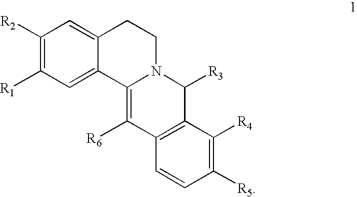
Wherein,
R₁ and R₂ are each independently H, OH, C₁-C₄ alkoxyl or C₁-C₄ acyloxy, or R₁ and R₂ are combined into —O—CH₂—O—;
R₃ is H, OH, C₁-C₄ alkoxyl, C₁-C₄ acyloxy, C₁-C₄ alkyl, C₁-C₄ haloalkyl or an aryl;
R₄ and R₅ are each independently H, OH, C₁-C₄ alkoxyl or C₁-C₄ acyloxy, or R₄ and R₅ are combined into —O—CH₂—O—; and
R₆ is H, OH, C₁-C₄ alkoxyl, C₁-C₄ acyloxy, C₁-C₄ alkyl, C₁-C₄ haloalkyl or an aryl.

4. The method according to claim 3, wherein the complication includes diabetes mellitus, adiposity, and fatty liver.

5. The method according to claim 3, wherein the compound of Formula 1 is one selected from the following compounds represented by Formulae 19 to 41: 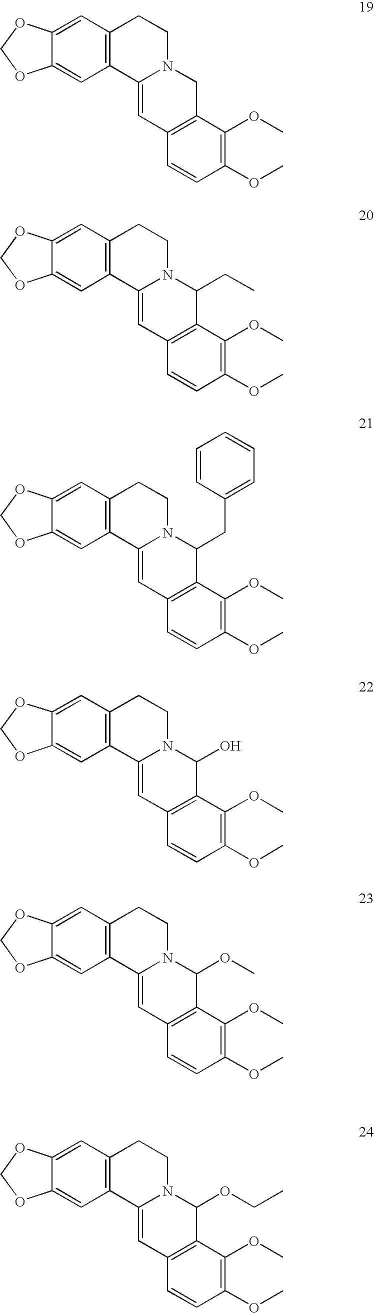 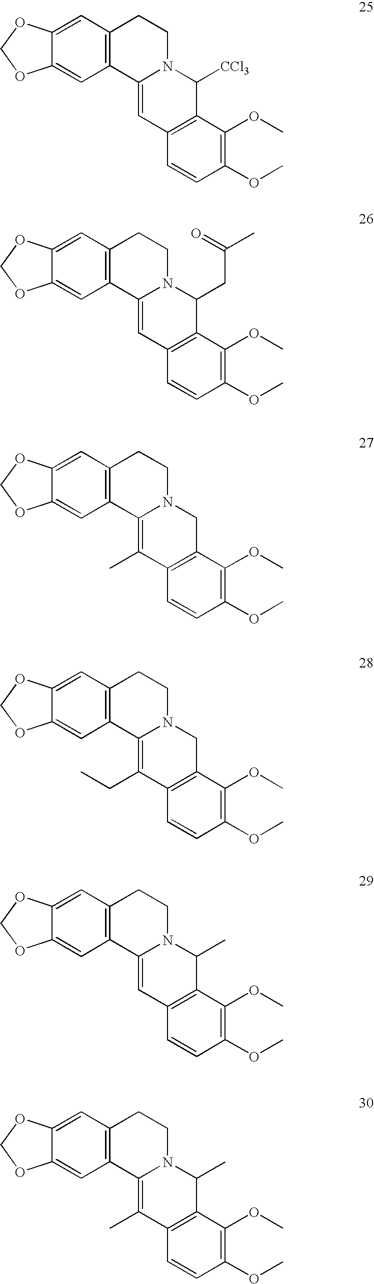 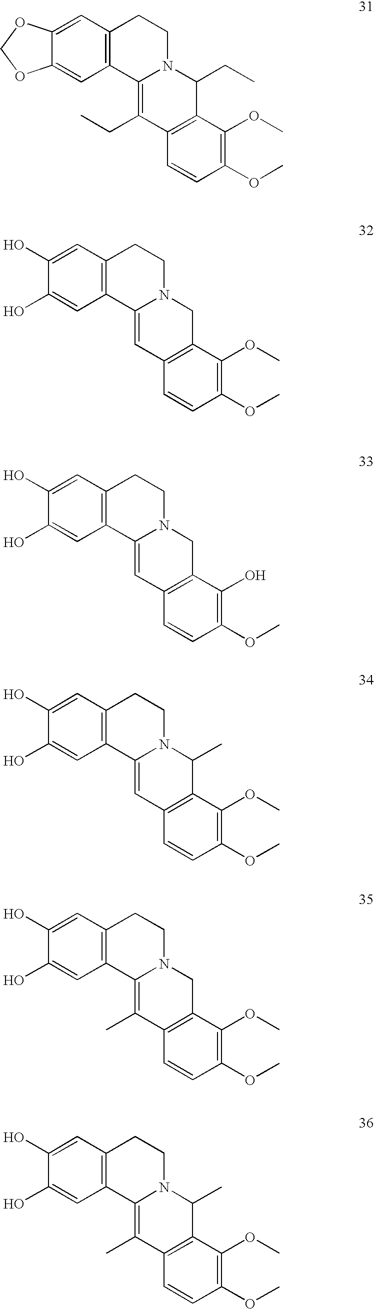 [chemical structure]
